(19) United States
(12) Patent Application Publication
(10) Pub. No.: US 2004/0006042 A1
(43) Pub. Date: Jan. 8, 2004

(54) Diaminopyrimidines and combination therapies effective for treatment of P-glycoprotein positive cancers

(76) Inventors: David Allen Berry, Ann Arbor, MI (US); Ellen Myra Dobrusin, Ann Arbor, MI (US); Judith Lynne Johnson-Philipsen, Ypsilanti, MI (US); Wayne Daniel Klohs, Ypsilanti, MI (US); Dennis Joseph McNamara, Ann Arbor, MI (US); Leslie Morton Werbel, Ann Arbor, MI (US)

Correspondence Address: WARNER-LAMBERT COMPANY, 2800 PLYMOUTH RD, ANN ARBOR, MI, 48105, US

(21) Appl. No.: 10/446,466

(22) Filed: May 28, 2003

Related U.S. Application Data
(63) Continuation of application No. 09/744,126, filed on Jan. 19, 2001.
Application No. 09/744,126 is a 371 of international application No. PCT/US99/13669, filed on Jun. 18, 1999.
(60) Provisional application No. 60/096,434, filed on Aug. 13, 1998.

(51) Int. Cl. A61K 31/7072; A61K 31/525; A61K 31/506; C 07D 4 3/04

(52) U.S. Cl. 514/50; 514/251; 514/252.14; 544/295

(57) ABSTRACT

The present invention provides compounds of Formula II:

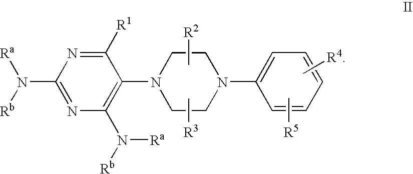

### CROSS-REFERENCE TO RELATED APPLICATIONS

This is a continuation of Ser. No. 09/744,126, filed Jan. 19, 2001, which is a 371 filing of PCT/US99/13669 filed Jun. 18, 1999, which claims priority to U.S. Provisional Serial No. 60/096,434 filed Aug. 13, 1998.

### FIELD OF THE INVENTION

Tumors that express P-glycoprotein demonstrate the phenomenon of multidrug resistance (MDR) and are resistant to many forms of chemotherapy. The present invention provides combination therapies including diaminopyrimidines that overcome P-glycoprotein-induced multidrug resistance in P-glycoprotein positive tumors both in vitro and in vivo. Also provided are pharmaceutical compositions and a method of treating cancer.

### BACKGROUND OF THE INVENTION

Randomized clinical trials of patients with colorectal cancer have clearly demonstrated that the combination of leucovorin (LV) and 5-fluorouracil (FU) significantly enhanced the response rate versus FU alone. LV/FU treatment of colorectal patients has become the standard chemotherapy for patients with this disease. The combination of methotrexate (MTX) and FU also resulted in improved response rates in patients with colorectal cancer compared to patients treated with FU alone. A three drug combination of MTX, FU and LV, however, did not result in increased activity because MTX competes with LV in the reduced folate polyglutamylation pathway and for transport into the cell.

Trimetrexate (TMQ), which is a lipophilic antifolate, does not use the reduced folate transport system for uptake and is not polyglutamylated. It has been demonstrated that in contrast to LV/FU/MTX, the three drug combination of LV/FU/TMQ demonstrated a high level of synergistic activity against a human lymphocytic leukemia cell line in vitro. Based on these observations, several clinical trials have been initiated in colon cancer with LV/FU/TMQ. In fact, it has been recently reported that the addition of TMQ to LV/FU resulted in a 20% response rate in colon cancer patients previously treated with FU. It was also recently reported that the combination of TMQ, FU and LV resulted in a 50% response rate in metastatic colorectal cancer patients.

Biochemically, LV potentiates FU cytotoxicity in vitro by increasing intracellular levels of N⁵,N¹⁰-methylenetetrahydrofolate and its polyglutamates and thereby stimulating and prolonging fluorodeoxyunridine monophosphate (FdUMP) binding to thymidylate synthase (TS). TMQ or MTX inhibit dihydrofolate reductase which blocks purine biosynthesis and in turn, results in increased levels of phosphoribosyl pyrophosphate (PRPP). The increased levels of PRPP result in an increase in FU nucleotides formed intracellularly by increasing incorporation of FU into RNA and increasing the levels of FdUMP, the inhibitor of TS. Moreover, TMQ or MTX inhibition of dihydrofolate reductase leads to the partial depletion of existing N⁵,N¹⁰-methylenetetrahydrofolate pools, resulting in increased levels of dihydrofolate polyglutamates, which in turn are also able to form potent complexes with TS and FdUMP.

The addition of TMQ to LV/FU chemotherapy regime to treat colorectal cancer suffers from one major drawback: almost 85% of all colorectal cancers overexpress P-glycoprotein, a transport protein found on the cell surface of many cancers that rapidly transports many anticancer agents including TMQ out of the cancer cell before it can exert any cytotoxic effect. The over-expression of P-glycoprotein in tumors such as colon results in a cellular phenotype known as multi-drug resistance or MDR. Therefore, in spite of the biochemical rationale and proven synergy of the combination of TMQ with LV/FU in cultured P-glycoprotein negative human leukemia cells, TMQ's effect with LV/FU is likely limited to those few colon cancers that do not express P-glycoprotein. New methods of treating cancer are desired.

U.S. Pat. No. 4,532,240, which is hereby incorporated by reference in its entirety, discloses certain substituted diarminopyrimidines having the general Formula I

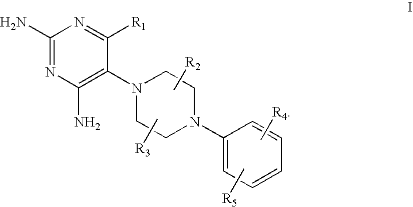

The compounds are disclosed to have antibacterial and antitumor activity.

### SUMMARY OF THE INVENTION

The present invention provides a method of treating cancer, the method comprising administering to a patient having cancer, a therapeutically effective amount of a combination of:

1) 5-fluorouracil;

2) leucovorin; and

3) a compound of Formula I

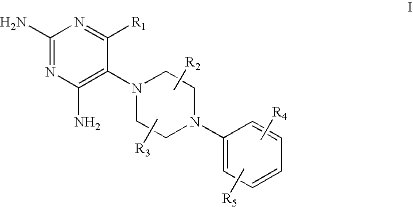

wherein

R₁ is hydrogen or alkyl of from one to six carbon atoms;

R₂ and R₃ are independently hydrogen or methyl;

R₄ and R₅ are independently:

hydrogen;

halogen;

nitro;

cyano;

trifluoromethyl;

hydroxyl;

alkyl of from one to six carbon atoms;

alkoxyl of from one to six carbon atoms;

alkanoyl of from one to six carbon atoms;

—NR₆R₇, where R₆ and R₇ are independently

hydrogen,

alkyl of from one to six carbon atoms,

alkanoyl of from one to six carbon atoms;

—COOR₈ where R₈ is

hydrogen,

a pharmaceutically acceptable metal cation,

a pharmaceutically acceptable amine cation,

alkyl of from one to six carbon atoms;

—CONR₉R₁₀ where R₉ and R₁₀ are independently

hydrogen,

alkyl of from one to six carbon atoms,

alkyl of from one to six carbon atoms, substituted with one or two carboxyl groups,

alkyl of from one to six carbon atoms, substituted with one or two carboxyl groups and one —OH, —SH, or —NH₂ group,

alkyl of from one to six carbon atoms, substituted with one or two carboalkoxy groups of from one to six carbon atoms,

alkyl of from one to six carbon atoms, substituted with one or two carboalkoxy groups of from one to six carbon atoms and one —OH, —SH, or —NH₂ group;

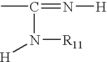

where R₁₁ is hydrogen, or alkyl of from one to six carbon atoms; or

—SO₂R₁₂ where R₁₂ is

hydroxyl,

alkyl of from one to six carbon atoms,

alkoxy of from one to six carbon atoms, or

—NR₁₃R₁₄ where R₁₃ and R₁₄ are independently hydrogen or alkyl of from one to six carbon atoms;

and the pharmaceutically acceptable salts thereof.

The present invention also provides a method of treating cancer, the method comprising administering to a patient having cancer, a therapeutically effective amount of a combination of:

1) a thymidylate synthase inhibitor; and

2) a compound of Formula I

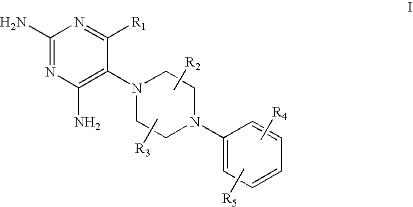

wherein

R₁ is hydrogen or alkyl of from one to six carbon atoms;

R₂ and R₃ are independently hydrogen or methyl;

R₄ and R₅ are independently:

hydrogen,

halogen,

nitro,

cyano,

trifluoromethyl,

hydroxyl,

alkyl of from one to six carbon atoms,

alkoxyl of from one to six carbon atoms,

alkanoyl of from one to six carbon atoms;

—NR₆R₇, where R₆ and R₇ are independently

hydrogen,

alkyl of from one to six carbon atoms,

alkanoyl of from one to six carbon atoms;

—COOR₈ where R₈ is

hydrogen,

a pharmaceutically acceptable metal cation,

a pharmaceutically acceptable amine cation,

alkyl of from one to six carbon atoms;

—CONR₉R₁₀ where R₉ and R₁₀ are independently hydrogen,

alkyl of from one to six carbon atoms,

alkyl of from one to six carbon atoms, substituted with one or two carboxyl groups,

alkyl of from one to six carbon atoms, substituted with one or two carboxyl groups and one —OH, —SH, or —NH₂ group,

alkyl of from one to six carbon atoms, substituted with one or two carboalkoxy groups of from one to six carbon atoms,

alkyl of from one to six carbon atoms, substituted with one or two carboalkoxy groups of from one to six carbon atoms and one —OH, —SH, or —NH₂ group;

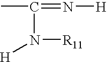

where R₁₁ is hydrogen, or alkyl of from one to six carbon atoms; or

—SO₂R₁₂ where R₁₂ is

hydroxyl,

alkyl of from one to six carbon atoms,

alkoxy of from one to six carbon atoms, or

—NR₁₃R₁₄ where R₁₃ and R₁₄ are independently hydrogen or alkyl of from one to six carbon atoms;

and the pharmaceutically acceptable salts thereof.

In a preferred embodiment of the methods, the cancer is colorectal cancer.

In a preferred embodiment of the method, the thymidylate synthase inhibitor is 2-({5-[Methyl-(2-methyl-4-oxo-3,4-dihydro-quinazolin-6-ylmethyl)-amino]-thiophene-2-carbonyl}-amino)-pentanedioic acid.

In another preferred embodiment of the method, the thymidylate synthase inhibitor is:

2-{4-[2-(2-Amino-4-oxo-4,7-dihydro-3H-pyrrolo[2,3-d]pyrimidin-5-yl)-ethyl]-benzoylamino}-pentanedioic acid;

N⁶-Methyl-N⁶-[4-(morpholine-4-sulfonyl)-benzyl]-benzo[cd]indole-2,6-diamine; compound with 3,4,5,6-tetrahydroxy-tetrahydro-pyran-2-carboxylic acid; or

2-Amino-6-methyl-5-(pyridin-4-ylsulfanyl)-3H-quinazolin-4-one.

In a more preferred embodiment of the method, the compounds of Formula I are:

4-[4-(2,4-Diamino-6-methyl-5-pyrimidinyl)-1-piperazinyl]benzoic acid;

4-[4-(2,4-Diamino-6-methyl-5-pyrimidinyl)- 1 -piperazinyl]benzoic acid ethyl ester;

5-[4-(4-Cyanophenyl)-1-piperazinyl]-6-methyl-2,4-pyrimidinediamine;

N-[4-[4-(2,4-Diamino-6-methyl-5-pyrimidinyl)-1-piperazinyl]-benzoyl]-L-glutamic acid;

N-[4-[4-(2,4-Diamino-6-methyl-5-pyrimidinyl)-1-piperazinyl]-benzoyl]-L-glutamic acid diethyl ester;

6-Methyl-5-[4-(4-nitrophenyl)-1-piperazinyl]-2,4-pyrimidinediamine;

5-[4-(4-Acetylphenyl)-1-piperazinyl]-6-methyl-2,4-pyrimidinediamine;

4-[4-(2,4-Diamino-6-methyl-pyrimidinyl)-1-piperazinyl]-N-methyl benzamide;

[4-(2,4-Diamino-6-methyl-5-pyrimidinyl)-1-piperazinyl]benzoyl]glycine;

N-[4-[4-(2,4-Diamino-6-methyl-5-pyrimidinyl)-1-piperazinyl]benzoyl]glycine methyl ester;

6-Methyl-5-[4-[4-(methylsulfonyl)phenyl]-1-piperazinyl]-2,4-pyrimidinediamine;

5-[4-(4-Cyanophenyl)-1-piperazinyl]-6-ethyl-2,4-pyrimidinediamine;

4-[4-(2,4-Diamino-6-ethyl-5-pyrimidinyl)-1-piperazinyl]benzoic acid;

N-[4-[4-(2,4-Diamino-6-ethyl-5-pyrimidinyl)-1-piperazinyl]benzoyl]-L-glutamic acid;

N-[4-[4-(2,4-Diamino-6-ethyl-5-pyrimidinyl)-1-piperazinyl]benzoyl]-L-glutamic acid diethyl ester;

5-[4-(4-Cyanophenyl)-1-piperazinyl]-6-propyl-2,4-pyrimidinediamine;

4-[4-(2,4-Diamino-6-propyl-5-pyrimidinyl)-1-piperazinyl]benzoic acid;

N-[4-[4-(2,4-Diamino-6-propyl-5-pyrimidinyl)-1-piperazinyl]benzoyl]-L-glutamic acid;

N-[4-[4-(2,4-Diamino-6-propyl-5-pyrimidinyl )-1-piperazinyl]benzoyl]-L-glutamic acid diethyl ester;

4-[4-(2,4-Diamino-6-propyl-5-pyrimidinyl)-1-piperazinyl]benzoic acid ethyl ester;

6-Methyl-5-(4-phenyl-1-piperazinyl)-2,4-pyrimidinediamine, hydrochloride;

5-[4-(4-Chlorophenyl)-1-piperazinyl]-6-methyl-2,4-pyrimidinediamine;

5-[4-(4-Aminophenyl-1-piperazinyl]-6-methyl-2,4-pyrimidinediamine;

N-[4-[4-(2,4-Diamino-6-methyl-5-pyrimidinyl)-1-piperazinyl]benzoyl]-glycine;

4- [4-(2,4-Diamino-6-methyl-5-pyrimidinyl)-1-piperazin yl]-N,N-dimethylbenzenesulfonamide; and

6-Methyl-5-[4-[4-trifluoromethyl)phenyl]-1-piperazinyl]-2,4-pyrimidinediamine.

In a preferred embodiment of the methods, the cancer is a P-glycoprotein positive cancer.

Also provided are compounds having Formula II

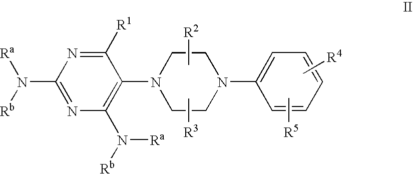

wherein

each n is independently 0 to 6;

R^{a} and R^{b} are independently hydrogen or C₁-C₆ alkyl;

R¹ is C₁-C₆ alkyl or

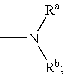

R² and R³ are independently hydrogen, —C₁-C₆ alkyl, —CH₂OH, or —OH;

R⁴ is hydrogen;

R⁵ is —(CH₂)ₙ—OH, —(CH₂)ₙNH₂,

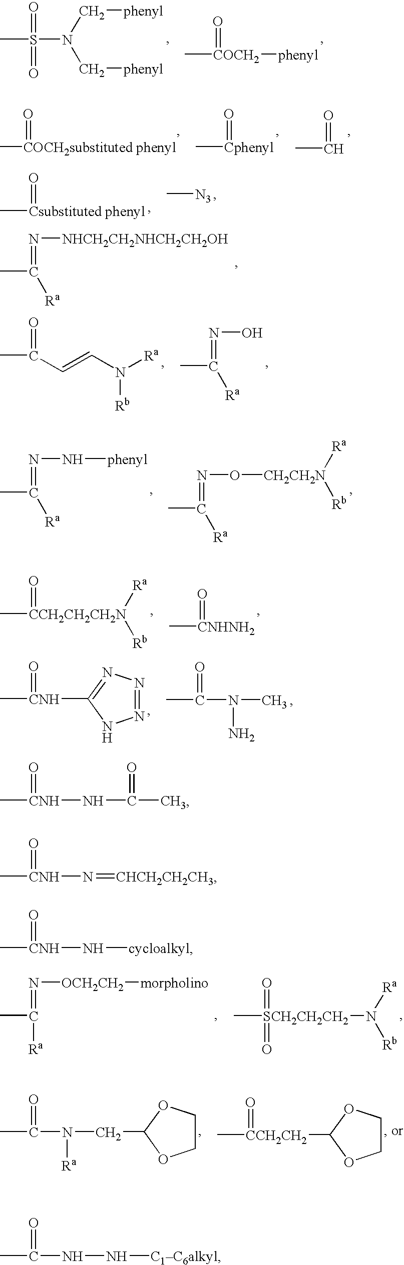

and the pharmaceutically acceptable salts thereof.

In a preferred embodiment of the method, R^{a} and R^{b} are independently hydrogen or methyl.

In another preferred embodiment, R¹ is methyl.

In still another preferred embodiment, R² and R³ are hydrogen.

In a more preferred embodiment,

R^{a} and R^{b} are independently hydrogen or methyl;

R¹ is methyl;

R² and R³ are hydrogen; and

R⁴ is hydrogen.

In a most preferred embodiment, the present invention provides the compounds:

4-[4-(2,4-Diamino-methyl-pyrimidin-5-yl)-piperazin-1-yl)-benzaldehyde;

4-[4-(2,4-Diamino-6-methyl-pyrimidin-5-yl)-piperazin-1-yl]-benzaldehyde O-(2-phenoxy-ethyl)-oxime monohydrochloride;

4-[4-(2,4-Diamino-6-methyl-pyrimidin-5-yl)-piperazin-1-yl]-benzaldehyde O-benzyl-oxime monohydrochloride;

4-[⁴-(2,4-Diamino-6-methyl-pyrimidin-5-yl)-piperazin-1-yl]-benzaldehyde O-(4-bromo-3-hydroxy-benzyl)-oxime monohydrochloride;

{4-[4-(2,4-Diamino-6-methyl-pyrimidin-5-yl)-piperazin-1-yl]-benzylideneaminooxy}-acetic acid phenethyl ester;

{4-[4-(2,4-Diamino-6-methyl-pyrimidin-5-yl)-piperazin-1-yl]-benzylideneaminooxy}-acetic acid monohydrochloride;

4-[4-(2,4-Diamino-6-methyl-pyrimidin-5-yl)-piperazin-1-yl]-benzaldehyde O-(3-phenyl-propyl)-oxime, monohydrochloride;

4-[4-(2,4-Diamino-6-methyl-pyrimidin-5-yl)-piperazin-1yl]-benzaldehyde O-(3-phenoxy-propyl)-oxime monohydrochloride;

4-[4-(2,4-Diamino-6-methyl-pyrimidin-5-yl)piperazin-1yl]-benzaldehyde, O-benzofuran-2-ylmethyl-oxime monooxalate;

1-[4-[4-(2,4-Diamino-6-methyl-5-pyrimidinyl)-1-piperazinyl]-4-(diethyl-amino)-1-butanone;

4-[4-(2,4-Diamino-6-methyl-5-pyrimidinyl)-1-piperazinyl]benzoic acid, hydrazide;

4-[4-(2,4-Diamino-6-methyl-pyrimidin-5-yl)-piperazin-1-yl]-benzaldehyde O-(2-amino-ethyl)-oxime, dihydrochloride;

4-[4-(2,4-Diamino-6-methyl-5-pryimidinyl)-1-piperazinyl]benzoic acid, 1-methylhydrazide;

4-[4-(2,4-Diamino-6-methyl-pyrimidin-5-yl)-piperazin-1-yl]-benzoic acid pentylidenehydrazide, hemiacetate;

4-[4-(2,4-Diamino-6-methyl-pryimidin-5-yl)-piperazin-1-yl]-benzoic acid, N′-pentyl-hydrazide;

4-[4-(2,4-Diamino-6-methyl-5-pryimidinyl)-1-piperazinyl]benzoic acid, 2-acetylhydrazide, hydrochloride;

6-Methyl-5-(4-{4-[1-(phenyl-hydrazono)-ethyl]-phenyl}-piperazin-1-yl)-pyrimidine-2,4-diamine;

Benzaldehyde, 4-[4-(2,4-diamino-6-methyl-5-pyrimidinyl)-1-piperazinyl]-,[2-[(2-hydroxyethyl)amino]ethyl]hydrazone, methanesulfonate;

Ethanone, 1-[4-(2,4-diamino-6-methyl-5-pyrimidinyl)-1-piperazinyl]phenyl]-, oxime, monohydrochloride;

1-{4-[4-(2,4-Diamino-6-methyl-pyrimidin-5-yl)-piperazin-1-yl]-phenyl}-ethanone O-(2-amino-ethyl)-oxime, hydrochloride;

4-[4-(2,4-Diamino-6-methyl-pyrimidin-5-yl)-piperazin-1-yl]-benzaldehyde O-(2-methylamino-ethyl)-oxime, dihydrochloride;

4-[4-(2,4-Diamino-6-methyl-pyrimidin-5-yl)-piperazin-1-yl]-benzoic acid, 2-cyclohexylhydrazide;

4-[4-(2,4-Diamino-6-methyl-pyrimidin-5-yl)-piperazin-1-yl]-benzaldehyde O-(2-morpholin-4-yl-ethyl)-oxime;

4-[4-(2,4-Diamino-6-methyl-pyrimidin-5-yl)-piperazin-1-yl]-benzaldehyde O-(2-diethylamino-ethyl)-oxime, dihydrochloride;

1-{4-4-(2,4-Diamino-6-methyl-pyrimidin-5-yl)-piperazin-1-yl]-phenyl}-ethanone O-(2-diethylamino-ethyl)-oxime, dihydrochloride;

5-{4-[4-(3-Dimethylamino-propane-1-sulfonyl)-phenyl]-piperazin-1-yl}-6-methyl-pyrimidine-2,4-diamine; and

1-{4-[4-(2,4-Diamino-6-methyl-5-pyrimidinyl)-piperazin-1-yl]-phenyl}-ethanone O-(2-methylamino-ethyl)-oxime, trihydrochloride.

Also provided is a method of treating cancer, the method comprising administering to a patient having cancer, a therapeutically effective amount of a combination of:

1) 5-fluorouracil;

2) leucovorin; and

3) a compound of Formula II.

Also provided is a method of treating cancer, the method comprising administering to a patient having cancer, a therapeutically effective amount of a combination of:

(1) a thymidylate synthase inhibitor; and

(2) a compound of Formula II.

In a preferred embodiment of the methods, the cancer is colorectal cancer.

In a preferred embodiment of the method, the thymidylate synthase inhibitor is 2-({5-[Methyl-(2-methyl-4-oxo-3,4-dihydro-quinazolin-6-ylmethyl)-amino]-thiophene-2-carbonyl}-amino)-pentanedioic acid.

In a preferred embodiment of the method, the thymidylate synthase inhibitor is:

2-{4-[2-(2-Amino-4-oxo-4,7-dihydro-3H-pyrrolo[2,3-d]pyrirnidin-5-yl)-ethyl]-benzoylamino}-pentanedioic acid;

N⁶-Methyl-N⁶-[4-(morpholine-4-sulfonyl)-benzyl]-benzo[cd]indole-2,6-diamine; compound with 3,4,5,6-tetrahydroxy-tetrahydro-pyran-2-carboxylic acid; or

2-Amino-6-methyl-5-(pyridin-4-ylsulfanyl)-3H-quinazolin-4-one.

In a more preferred embodiment of the methods, the cancer is a P-glycoprotein positive cancer.

Also provided is a pharmaceutical composition that comprises a compound of Formula II.

Also provided is a method of treating cancer, the method comprising administering to a patient having cancer, a therapeutically effective amount of a combination of:

1) 5-fluorouracil;

2) leucovorin; and

3) a compound of Formula II

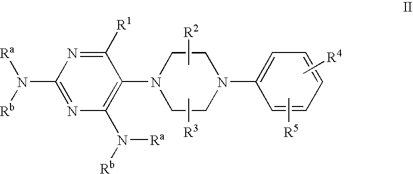

wherein

each R^{a} and R^{b} are independently hydrogen, C₁-C₆ alkyl or

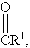

or C₁-C₆ cycloalkyl;

each n is independently 0 to 5;

R¹ is hydrogen, C₁-C₆ alkyl, or —NR^{a}R^{b};

R² and R³ are independently hydrogen, C₁-C₆ alkyl, —CH₂OH, or —OH;

R⁴ is hydrogen, halogen, C₁-C₆ alkyl, —NO₂, —CN, CF₃, —OH, —OC₁-C₆ alkyl,

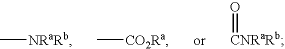

R⁵ is hydrogen, —CO₂R^{a},

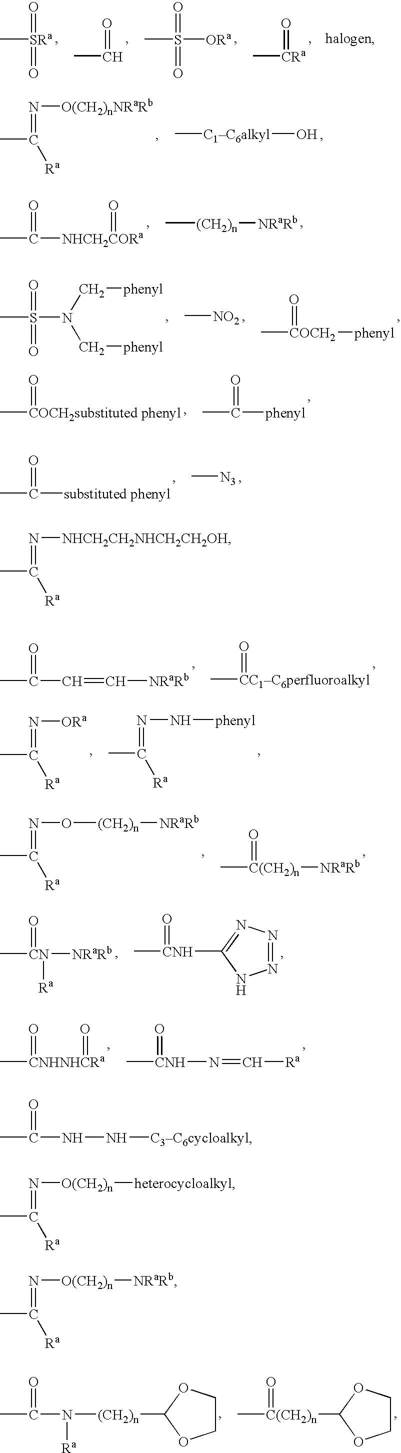

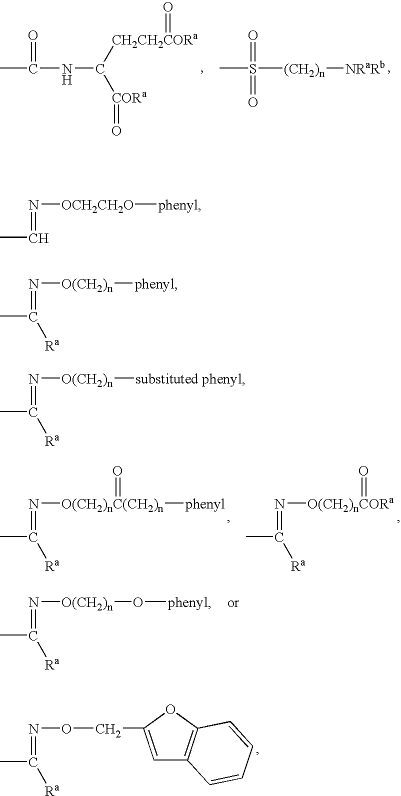

and the pharmaceutically acceptable salts thereof.

Also provided is a method of treating cancer, the method comprising administering to a patient having cancer, a therapeutically effective amount of a combination of:

1) a thymidylate synthase inhibitor; and

2) a compound of Formula II

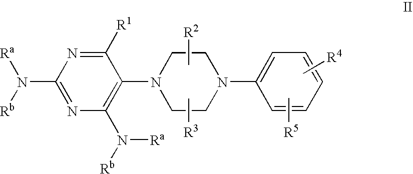

wherein

each R^{a} and R^{b} are independently hydrogen, C₁-C₆ alkyl,

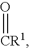

or C₁-C₆ cycloalkyl;

each n is independently 0 to 5;

R¹ is hydrogen C₁-C₆ alkyl, or —NR^{a}R^{b};

R² and R³ are independently hydrogen, C₁-C₆ alkyl, —CH₂OH, or —OH;

R⁴ is hydrogen, halogen, C₁-C₆ alkyl, —NO₂, —CN, CF₃, —OH, —OC₁-C₆ alkyl,

—NR^{a}R^{b}, —CO₂R^{a}, or

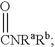

R⁵ is hydrogen, —CO₂R^{a},

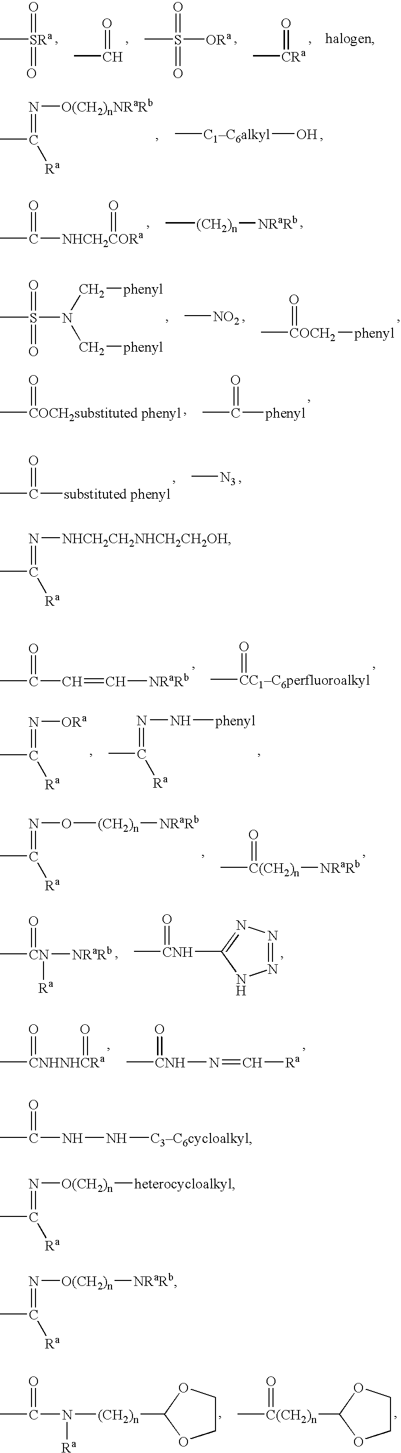

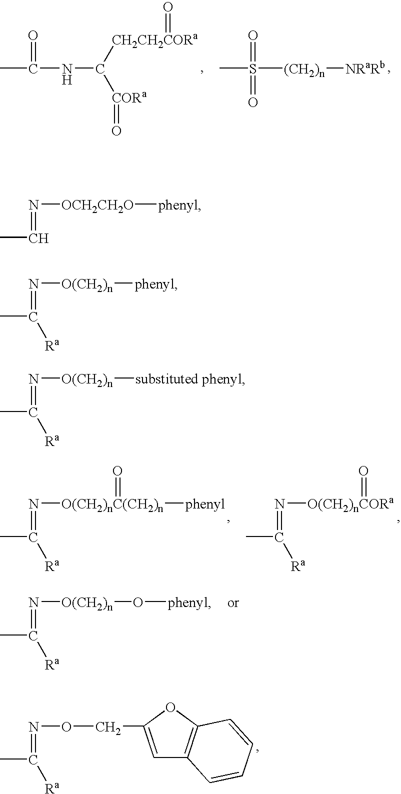

and the pharmaceutically acceptable salts thereof.

In a preferred embodiment, the cancer is colorectal cancer.

In another preferred embodiment, the thymidylate synthase inhibitor is 2-({5-[Methyl-(2-methyl-4-oxo-3,4-dihydro-quinazolin-6-ylmethyl)-amino]-thiophene-2-carbonyl}-amino)-pentanedioic acid.

In another preferred embodiment, the thymidylate synthase inhibitor is:

2-{4-[2-(2-Amino-4-oxo-4,7-dihydro-3H-pyrrolo[2,3-d]pyrimidin-5-yl)-ethyl]-benzoylamino}-pentanedioic acid;

N⁶-Methyl-N⁶-[4-(morpholine-4-sulfonyl)-benzyl]-benzo[cd]indole-2,6-diamine; compound with 3,4,5,6-tetrahydroxy-tetrahydro-pyran-2-carboxylic acid; or

2-Amino-6-methyl-5-(pyridin-4-ylsulfanyl)-3H-quinazolin-4-one.

In still another preferred embodiment, the cancer is a P-glycoprotein positive cancer.

### DETAILED DESCRIPTION OF THE INVENTION

The term “alkyl”0 means a straight or branched hydrocarbon having from 1 to 6 carbon atoms and includes, for example, methyl, ethyl, n-propyl, isopropyl, n-butyl, sec-butyl, isobutyl, tert-butyl, n-pentyl, n-hexyl, and the like. The alkyl group can also be substituted with one or more of the substituents listed below for aryl.

The term “cycloalkyl” means a saturated hydrocarbon ring which contains from 3 to 7 carbon atoms, for example, cyclopropyl, cyclobutyl, cyclopentyl, cyclohexyl, adamantyl, and the like.

The term “aryl” means an aromatic ring which is a phenyl, 5-fluorenyl, 1-naphthyl, or 2-naphthyl group, unsubstituted or substituted by 1 to 3 substituents selected from alkyl, O-alkyl and S-alkyl, OH, SH, F, —CN, Cl, Br, I, CF₃, NO₂, NH₂, —CN, —NH(C₁-C₆alkyl), —N(C₁-C₆alkyl)₂, —CO₂H, and —CO₂C₁-C₆ alkyl.

The term “heteroaryl” means an aromatic ring containing one or more heteroatoms. Examples of heteroaryl radicals include thienyl, furanyl, pyrrolyl, pyridyl, imidazoyl, or indolyl group, substituted or unsubstituted by 1 or 2 substituents from the group of substituents described above for aryl. Examples of heteroatoms include nitrogen, oxygen, sulfur, and phosphorus.

The symbol “-” means a bond.

The term “patient” means all animals including humans. Examples of patients include humans, cows, dogs, cats, goats, sheep, and pigs.

A “therapeutically effective amount” is an amount of a compound of the present invention that when administered to a patient ameliorates a symptom of cancer. A therapeutically effective amount of a compound of the present invention can be easily determined by one skilled in the art by administering a quantity of a compound to a patient and observing the result. In addition, those skilled in the art are familiar with identifying patients having cancer.

The term “cancer” includes, but is not limited to, the following cancers:

breast;

ovary;

cervix;

prostate;

testis;

esophagus;

glioblastoma;

neuroblastoma;

stomach;

skin, keratoacanthoma;

lung, epidermoid carcinoma, large cell carcinoma, adenocarcinoma;

bone;

colon, adenocarcinoma, adenoma;

pancreas, adenocarcinoma;

thyroid, follicular carcinoma, undifferentiated carcinoma, papillary carcinoma;

seminoma;

melanoma;

sarcoma;

bladder carcinoma;

liver carcinoma and biliary passages;

kidney carcinoma;

myeloid disorders;

lymphoid disorders, Hodgkins disease, hairy cells;

buccal cavity and pharynx (oral), lip, tongue, mouth, pharynx;

small intestine;

colon-rectum, large intestine, rectum;

brain and central nervous system; and leukemia.

Preferred cancers that can be treated by the present invention include colorectal, breast, colon, rectal, gastric, pancreatic, and liver cancer. A more preferred cancer to be treated is colorectal cancer. In a most preferred embodiment, the cancer is a P-glycoprotein positive cancer.

The term “P-glycoprotein positive cancer” means cancers that overexpress the drug resistance protein, P-glycoprotein P-glycoprotein positive cancers show the property of multidrug resistance.

One skilled in the art can determine if a cancer is P-glycoprotein positive by standard immunohistochemistry techniques using commercially available antibodies to P-glycoprotein.

The term “pharmaceutically acceptable salts, esters, amides, and prodrugs” as used herein refers to those carboxylate salts, amino acid addition salts, esters, amides, and prodrugs of the compounds of the present invention which are, within the scope of sound medical judgment, suitable for use in contact with the tissues of patients without undue toxicity, irritation, allergic response, and the like, commensurate with a reasonable benefit/risk ratio, and effective for their intended use, as well as the zwitterionic forms, where possible, of the compounds of the invention. The term “salts” refers to the relatively non-toxic, inorganic and organic acid addition salts of compounds of the present invention. These salts can be prepared in situ during the final isolation and purification of the compounds or by separately reacting the purified compound in its free base form with a suitable organic or inorganic acid and isolating the salt thus formed. Representative salts include the hydrobromide, hydrochloride, sulfate, bisulfate, nitrate, acetate, oxalate, valerate, oleate, palmitate, stearate, laurate, borate, benzoate, lactate, phosphate, tosylate, citrate, maleate, fumarate, succinate, tartrate, naphthylate mesylate, glucoheptonate, lactobionate and laurylsulphonate salts, and the like. These may include cations based on the alkali and alkaline earth metals, such as sodium, lithium, potassium, calcium, magnesium and the like, as well as non-toxic ammonium, quaternary ammonium, and amine cations including, but not limited to ammonium, tetramethylammonium, tetraethylammonium, methylamine, dimethylamine, trimethylamine, triethylamine, ethylamine, and the like. (See, for example, Berge S. M. et al., “Pharmaceutical Salts,” J. Pharm. Sci., 1977;66:1-19 which is incorporated herein by reference.)

Examples of pharmaceutically acceptable, non-toxic esters of the compounds of this invention include C₁-C₆ alkyl esters wherein the alkyl group is a straight or branched chain. Acceptable esters also include C₅-C₇ cycloalkyl esters as well as arylalkyl esters such as, but not limited to benzyl. C₁-C₄ alkyl esters are preferred. Esters of the compounds of the present invention may be prepared according to conventional methods.

Examples of pharmaceutically acceptable, non-toxic amides of the compounds of this invention include amides derived from ammonia, primary C₁-C₆ alkyl amines and secondary C₁-C₆ dialkyl amines wherein the alkyl groups are straight or branched chain. In the case of secondary amines the amine may also be in the form of a 5- or 6-membered heterocycle containing one nitrogen atom. Amides derived from ammonia, C₁-C₃ alkyl primary amines and C₁-C₂ dialkyl secondary amines are preferred. Amides of the compounds of the invention may be prepared according to conventional methods.

The term “prodrug” refers to compounds that are rapidly transformed in vivo to yield the parent compound of the above formulae, for example, by hydrolysis in blood. A thorough discussion is provided in T. Higuchi and V. Stella, “Pro-drugs as Novel Delivery Systems,” Vol. 14 of the A.C.S. Symposium Series, and in Bioreversible Carriers in Drug Design, ed. Edward B. Roche, American Pharmaceutical Association and Pergamon Press, 1987, both of which are hereby incorporated by reference.

The compounds of the present invention can be administered to a patient alone or as part of a composition that contains other components such as excipients, diluents, and carriers, all of which are well-known in the art. The compositions can be administered to humans and animals either orally, rectally, parenterally (intravenously, intramuscularly, or subcutaneously), intracisternally, intravaginally, intraperitoneally, intravesically, locally (powders, ointments, or drops), or as a buccal or nasal spray.

Compositions suitable for parenteral injection may comprise physiologically acceptable sterile aqueous or nonaqueous solutions, dispersions, suspensions or emulsions, and sterile powders. for reconstitution into sterile injectable solutions or dispersions. Examples of suitable aqueous and nonaqueous carriers, diluents, solvents or vehicles include water, ethanol, polyols (propyleneglycol, polyethyleneglycol, glycerol, and the like), suitable mixtures thereof, vegetable oils (such as olive oil), and injectable organic esters such as ethyl oleate. Proper fluidity can be maintained, for example, by the use of a coating such as lecithin, by the maintenance of the required particle size in the case of dispersions and by the use of surfactants.

These compositions may also contain adjuvants such as preserving, wetting, emulsifying, and dispensing agents. Prevention of the action of microorganisms can be ensured by various antibacterial and antifungal agents, for example, parabens, chlorobutanol, phenol, sorbic acid, and the like. It may also be desirable to include isotonic agents, for example sugars, sodium chloride, and the like. Prolonged absorption of the injectable pharmaceutical form can be brought about by the use of agents delaying absorption, for example, aluminum monostearate and gelatin.

Solid dosage forms for oral administration include capsules, tablets, pills, powders, and granules. In such solid dosage forms, the active compound is admixed with at least one inert customary excipient (or carrier) such as sodium citrate or dicalcium phosphate or (a) fillers or extenders, as for example, starches, lactose, sucrose, glucose, mannitol, and silicic acid; (b) binders, as for example, carboxymethylcellulose, alignates, gelatin, polyvinylpyrrolidone, sucrose, and acacia; (c) humectants, as for example, glycerol; (d) disintegrating agents, as for example, agar-agar, calcium carbonate, potato or tapioca starch, alginic acid, certain complex silicates, and sodium carbonate; (e) solution retarders, as for example paraffin; (f) absorption accelerators, as for example, quaternary ammonium compounds; (g) wetting agents, as for example, cetyl alcohol and glycerol monostearate; (h) adsorbents, as for example, kaolin and bentonite; and (i) lubricants, as for example, talc, calcium stearate, magnesium stearate, solid polyethylene glycols, sodium lauryl sulfate, or mixtures thereof. In the case of capsules, tablets, and pills, the dosage forms may also comprise buffering agents.

Solid compositions of a similar type may also be employed as fillers in soft and hard-filled gelatin capsules using such excipients as lactose or milk sugar as well as high molecular weight polyethyleneglycols, and the like.

Solid dosage forms such as tablets, dragees, capsules, pills, and granules can be prepared with coatings and shells, such as enteric coatings and others well-known in the art. They may contain opacifying agents, and can also be of such composition that they release the active compound or compounds in a certain part of the intestinal tract in a delayed manner. Examples of embedding compositions which can be used are polymeric substances and waxes. The active compounds can also be in micro-encapsulated form, if appropriate, with one or more of the above-mentioned excipients.

Liquid dosage forms for oral administration include pharmaceutically acceptable emulsions, solutions, suspensions, syrups, and elixirs. In addition to the active compounds, the liquid dosage forms may contain inert diluents commonly used in the art, such as water or other solvents, solubilizing agents and emulsifiers, as for example, ethyl alcohol, isopropyl alcohol, ethyl carbonate, ethyl acetate, benzyl alcohol, benzyl benzoate, propyleneglycol, 1,3-butyleneglycol, dimethylformamide, oils, in particular, cottonseed oil, groundnut oil, corn germ oil, olive oil, castor oil and sesame oil, glycerol, tetrahydrofurfuryl alcohol, polyethyleneglycols and fatty acid esters of sorbitan or mixtures of these substances, and the like.

Besides such inert diluents, the composition can also include adjuvants, such as wetting agents, emulsifying and suspending agents, sweetening, flavoring, and perfuming agents.

Suspensions, in addition to the active compounds, may contain suspending agents, as for example, ethoxylated isostearyl alcohols, polyoxyethylene sorbitol and sorbitan esters, microcrystalline cellulose, aluminum metahydroxide, bentonite, agar-agar and tragacanth, or mixtures of these substances, and the like.

Compositions for rectal administrations are preferably suppositories which can be prepared by mixing the compounds of the present invention with suitable non-irritating excipients or carriers such as cocoa butter, polyethyleneglycol, or a suppository wax, which are solid at ordinary temperatures but liquid at body temperature and therefore, melt in the rectum or vaginal cavity and release the active component.

Dosage forms for topical administration of a compound of this invention include ointments, powders, sprays, and inhalants. The active component is admixed under sterile conditions with a physiologically acceptable carrier and any preservatives, buffers, or propellants as may be required. Ophthalmic formulations, eye ointments, powders, and solutions are also contemplated as being within the scope of this invention.

The combination therapies of the present invention can be administered to a patient in a number of ways. For example, the different therapeutic agents can be given in a single dosage form such as together in a tablet, or in separate dosage forms, such as each agent in a separate tablet. In addition, when administered separately, a therapeutic agent may be given a certain time prior to the other agents. For example, the thymidylate synthase inhibitor may be given before a compound of Formula I or Formula II.

The administration of the therapeutic agent should be optimized so that the combination of agents is delivered to the cancer cells so that a synergistic cytotoxic effect is accomplished.

The compounds of the present invention can be administered to a patient at dosage levels in the range of about 0.1 to about 2,000 mg per day. For a normal human adult having a body weight of about 70 kilograms, a dosage in the range of about 0.01 to about 100 mg per kilogram of body weight per day is preferable. The specific dosage used, however, can vary. For example, the dosage can depended on a numbers of factors including the requirements of the patient, the severity of the condition being treated, and the pharmacological activity of the compound being used. The determination of optimum dosages for a particular patient is well known to those skilled in the art.

The compounds of the present invention can exist in different stereoisomeric forms by virtue of the presence of asymmetric centers in the compounds. It is contemplated that all stereoisomeric forms of the compounds as well as mixtures thereof, including racemic mixtures, form part of this invention.

In addition, the compounds of the present invention can exist in unsolvated as well as solvated forms with pharmaceutically acceptable solvents such as water, ethanol, and the like. In general, the solvated forms are considered equivalent to the unsolvated forms for the purposes of the present invention.

All references cited herein, including patents, are hereby incorporated by reference.

The examples presented below are intended to illustrate particular embodiments of the invention, and are not intended to limit the scope of the specification or the claims in any way. The starting materials for the specific syntheses below can be obtained in accordance with U.S. Pat. No. 4,532,240 which has been incorporated by reference.

### EXAMPLE 1

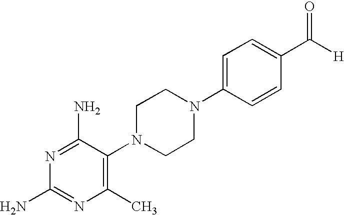

4-[4-(2,4-Diamino-methyl-pyrimidin-5-yl)-(piperazin-1-yl)-benzaldehyde

A mixture of 7.07 g (0.008 mol) of 6-methyl-5-(piperazin-1-yl)-6-methyl-pyrimidine-2,4-diamine, 1.55 mL (0.014 mol) of 4-fluorobenzaldehyde, 1.5 g of potassium fluoride, 1.52 g (0.0104 mol) of potassium carbonate, and 75 mL of dimethylsulfoxide was heated at 90° C. for 16 hours. The mixture was cooled to room temperature and filtered to collect 4.36 g of the title compound. MS (APCI+) 313.2 m/z, 100%. Recrystallization of a 1 g portion of the crude product from dimethylformamide gave 0.93 g of the title compound.

MS (APCI+) 313.2 m/z, 100%.

Analysis calculated for C₁₆H₂₀N₆O:

C, 61.52; H, 6.45; N, 26.90.

Found: C, 61.49; H, 6.53; N, 26.88.

### EXAMPLE 2

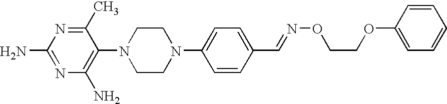

4-[4-(2,4-Diamino-6-methyl-pyrimidin-5-yl)-piperazin-1-yl]-benzaldehyde O-(2-phenoxy-ethyl)-oxime monohydrochloride

A mixture of 0.300 g (0.961 mmol) of 4-[4-(2,4-diamino-methyl-pyrimidin-5-yl)-piperazin-1 yl)-benzaldehyde and 0.293 g (1.923 mmol) of 2-phenoxy-ethyl-hydroxylamine in 25 mL of anhydrous ethanol was heated to reflux. After 40 minutes, 0.320 mL of a 6.5 M HCl/isopropanol solution was added to the mixture. Immediately the mixture frothed and turned from a yellow suspension into a white suspension. After refluxing for 8 hours, the mixture was cooled to room temperature, filtered, and the solid was rinsed with small portions of anhydrous ethanol. Upon drying the resulting solid, 0.41 g of the title compound was obtained as a white solid, mp >280° C.

MS (APCI M+) 310 m/z, 65%; 448 m/z, 100%.

Analysis calculated for C₂₄H₂₉N₇O₂·1.30 HCl·1.67 H₂O:

C, 54.91; H, 6.46; N, 18.67; Cl, 8.78.

Found: C, 54.90; H, 6.06; N, 18.74; Cl, 8.63.

### EXAMPLE 3

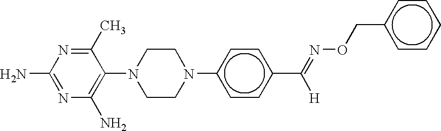

4-[4-(2,4-Diamino-6-methyl-pyrimidin-5-yl)-piperazin-1-yl]-benzaldehyde O-benzyl-oxime monohydrochloride

A mixture of 0.300 g (0.961 mmol) of 4-[4-(2,4-diamino-methyl-pyrimidin-5-yl)-piperazin-1-yl)-benzaldehyde, 0.260 g (1.634 mmol) of benzyl-hydroxylamine monohydrochloride, and 25 mL of anhydrous ethanol was heated and maintained under reflux. After 30 minutes, the mixture had turned from a yellow suspension into a white suspension. After 9 hours of reflux, the mixture was cooled to room temperature, filtered, and the solid was rinsed with small portions of anhydrous ethanol. Upon drying the resulting solid, 0.39 g of the title compound was obtained as a white solid, mp 280-281° C., decomposition.

MS (APCI M+) 310.2 m/z, 100%; 418.3 m/z, 50%.

Analysis calculated for C₂₄H₂₉N₇O₂·1.30 HCl·1.67 H₂O:

C, 58.44; H, 6.45; N, 20.74; Cl—, 7.05.

Found: C, 58.80; H, 6.24; N, 20.92; Cl—, 6.80.

### EXAMPLE 4

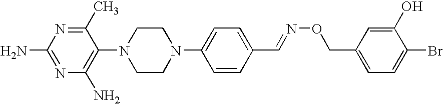

4-[4-(2,4-Diamino-6-methyl-pyrimidin-5-yl)-piperazin-1-yl]-benzaidehyde O-(4-bromo-3-hydroxy-benzyl)-oxime monohydrochloride

A mixture of 0.217 g (0.696 mmol) of 4-[4-(2,4-diamino-methyl-pyrimidin-5-yl)-piperazin-1-yl)-benzaldehyde, 0.258 g (1.18 mmol) of 5-aminooxymethyl-2-bromo-phenol, and 25 mL of anhydrous ethanol was heated and maintained under reflux. After 10 minutes, 0.320 mL of a 6.5 M HCl/isopropanol solution was added to the mixture. Immediately, the mixture frothed and turned from a yellow solid suspension into a white solid suspension. After refluxing for 1.5 hours, the mixture was cooled to room temperature and filtered, rinsed with small portions of anhydrous ethanol, and dried to afford 0.238 g of the title compound as a white solid, mp 253-255° C.

MS (APCI M+) 310 m/z, 100%; 512 m/z, 50%.

Analysis calculated for C₂₃H₂₆N₇O₂Br·1.94 HCl·0.23 H₂O:

C, 47.04; H, 4.87; N, 16.69; Cl—, 11.71.

Found: C, 46.91; H, 5.21; N, 16.42; Cl—, 11.33.

### EXAMPLE 5

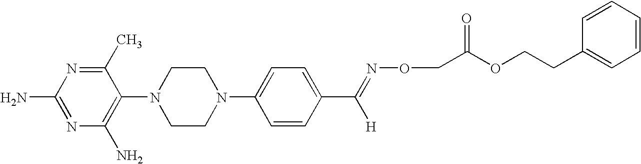

{4-[4-(2,4-Diamino-6-methyl-pyrimidin-5-yl)-piperazin-1yl]-benzylideneaminooxy}-acetic acid phenethyl ester

A mixture of 0.200 g (0.641 mmol) of 4-[4-(2,4-diamino-methyl-pyrimidin-5-yl)-piperazin-1-yl)-benzaldehyde, 0.252 g (1.09 mmol) of aminoxy-acetic acid phenethyl ester monohydrochloride, and 25 mL of anhydrous ethanol was heated and maintained under reflux. After 10 minutes, 0.320 mL of a 6.5 M HCl/isopropanol solution was added to the mixture. Immediately, the mixture turned from a yellow suspension into a white suspension. After refluxing for 1.5 hours, the mixture was cooled to room temperature, then was placed in the refrigerator for 20 minutes. The suspension was filtered, and the solid collected was washed with small portions of anhydrous ethanol to afford, after drying, 0.254 g of the title compound as a white solid, mp 255-257° C.

MS (APCI M+) 310.2 m/z, 95%; 490.3 m/z, 100%.

### EXAMPLE 6

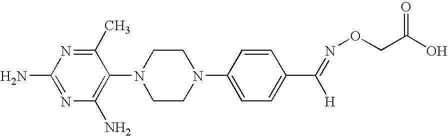

{4-[4-(2,4-Diamino-6-methyl-pyrimidin-5-yl)-piperazin-1-yl]-benzylideneaminooxy}-acetic acid monohydrochloride

A mixture of 0.300 g (0.964 mmol) of 4-[4-(2,4-diamino-methyl-pyrimidin-5-yl)-piperazin-1-yl)-benzaldehyde, 0.179 g (1.64 mmol) of aminooxy-acetic acid hemihydrochloride, and 25 mL of anhydrous ethanol was heated and maintained under reflux. The mixture turned from a yellow suspension into a white suspension. After refluxing for 1 hour, the mixture was cooled to room temperature and placed in the refrigerator. The mixture was filtered, and the resulting solid was washed with small portions of anhydrous ethanol to afford, after drying, 0.370 g of the title compound as a white solid.

MS (APCI M+) 310.2 m/z, 100%; 386.2 m/z 85%.

Analysis calculated for C₁₈H₂₃N₇O₃·0.74 HCl·0.47 H₂O:

C, 51.37; H, 5.91; N, 23.30; Cl—, 6.23.

Found: C, 51.68; H, 5.86; N, 23.51; Cl—, 5.98.

### EXAMPLE 7

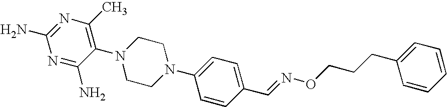

4-[4-(2,4-Diamino-6-methyl-pyrimidin-5-yl)-piperazin-1-yl]-benzaldehyde O-(3-phenyl-propyl)-oxime, monohydrochloride

A mixture of 0.245 g (0.784 mmol) of 4-[4-(2,4-diamino-methyl-pyrimidin-5-yl)-piperazin-1-yl)-benzaldehyde, 0.250 g (1.33 mmol) of 3-phenyl-propyl-hydroxylamine monohydrochloride, and 10 mL of anhydrous ethanol was heated and maintained under reflux. In a few minutes the mixture turned from a yellow solid suspension into a white solid suspension. After 20 minutes of reflux, the mixture thickened and was allowed to cool to room temperature. The mixture was further cooled in the refrigerator then filtered, washing with small portions of anhydrous ethanol, to obtain a white sticky solid. The solid was transferred to a vial and placed in an oven under vacuum overnight. To the resulting solid was added 10 mL water and 10 mL dichloromethane, and the pH of the aqueous layer was adjusted to pH 8 using 0.1 M NaOH. The aqueous layer was separated, and the cloudy organic portion was washed with brine. The organic portion was separated and concentrated in vacuo to leave 0.173 g of the title compound as a white solid, mp >280° C.

MS (APCI M+) 310.2 m/z, 65%; 446.4 m/z, 100%.

Analysis calculated for C₂₅H₃₁N₇O·1.03 HCl·0.07 H₂O:

C, 61.93; H, 6.95; N, 20.22; Cl—, 7.60.

Found: C, 61.63; H, 6.64; N, 20.34; Cl—, 7.32.

### EXAMPLE 8

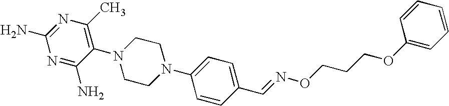

4-[4-(2,4-Diamino-6-methyl-pyrimidin-5-yl)-piperazin-1yl]-benzaldehyde O-(3-phenoxy-propyl)-oxime monohydrochloride

A mixture of 0.300 g (0.962 mmol) of 4-[4-(2,4-diamino-methyl-pyrimidin-5-yl)-piperazin-1-yl)-benzaldehyde, 0.333 g (1.63 mmol) of 3-phenoxy-propyl-hydroxylamine monohydrochloride, and 13 mL of anhydrous ethanol was heated and maintained under reflux. In a few minutes, the mixture turned from a yellow solid suspension into a white solid suspension. After 20 minutes of reflux, the mixture thickened and was allowed to cool to room temperature and then stored in the refrigerator overnight. The mixture was filtered, and the solid was washed with small portions of anhydrous ethanol to afford a white solid. The filtrate was refrigerated to induce precipitation of a second crop which was collected and combined with the first crop for drying in vacuo. To the combined solids were added 20 mL water and 20 mL dichloromethane and the pH of the aqueous layer was adjusted to pH 8 using 0.1 M NaOH. The aqueous layer was separated, and the organic phase was washed with brine. The organic portion was separated and concentrated in vacuo to afford a white solid which was contaminated with starting aldehyde. This material was retreated with 0.069 g of 3-phenoxy-propyl-hydroxylamine hydrochloride and 0.017 mL of anhydrous ethanol at room temperature for 96 hours to afford after workup the title compound as a white solid, 0.230 g, mp >280° C.

MS (APCI M+) 462 m/z, 100%.

Analysis calculated for C₂₅H₃₁N₇O₂·0.91 HCl·0.51 H₂O:

C, 59.59; H, 6.59; N, 19.46; Cl—, 6.40.

Found: C, 59.57; H, 6.36; N, 19.48; Cl—, 6.66.

### EXAMPLE 9

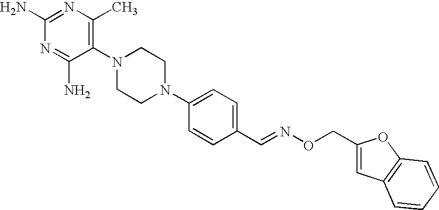

4-[4-(2,4-Diamino-6-methyl-pyrimidin-5-yl)piperazin-1yl]-benzaldehyde, O-benzofuran-2-ylmethyl-oxime monooxalate

A mixture of 0.250 g (0.801 mmol) of 4-[4-(2,4-diamino-methyl-pyrimidin-5-yl)-piperazin-1-yl)-benzaldehyde, 0.345 g (1.36 mmol) of benzofuran-2-ylmethyl-hydroxylamine monooxalate, and 10 mL of anhydrous ethanol was heated and maintained under reflux for 7.5 hours. The mixture was cooled to room temperature, filtered, and the solid washed with small portions of anhydrous ethanol. The resulting white solid was dried in an oven under vacuum overnight and recrystallized from hot isopropanol/water. The mixture was filtered while hot leaving 0.027 of undissolved solid. The filtrate was placed in the refrigerator for 30 minutes, then was concentrated to dryness in vacuo to give an orange solid. Isopropanol (10 mL) was added to the orange solid, and the mixture was put in the refrigerator overnight. The mixture was again filtered, rinsed sequentially with isopropanol, ethanol, and ether to afford 0.155 g of the title compound as an orange solid.

MS (APCIM+) 131 m/z, 45%; 310 m/z, 100%; 458.3 m/z, 100%.

Analysis calculated for C₂₅H₂₇N₇O₂·0.88 C₂H₂O₄·1.90 H₂O:

C, 56.29; H, 5.75; N, 17.17.

Found: C, 56.12; H, 5.35; N, 17.55.

### EXAMPLE 10

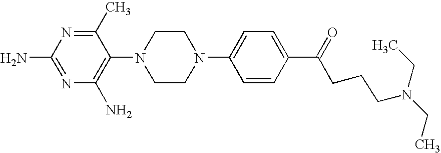

1-[4-[4-(2,4-Diamino-6-methyl-5-pyrimidinyl)-1-piperazinyl]-4-(diethyl-amino)-1-butanone

A suspension of 2.92 g (0.014 mol) of 6-methyl-5-(1-piperazinyl)-2,4-pyrimidinediamine, 3.38 g (0.0142 mol) of 4-(diethylamino)-1-(4-fluorophenyl)-l-butanone, 0.84 g (0.0145 mol) of dry KF, and 2.00 g (0.0145 mol) of potassium carbonate in 75 mL of dimethyl sulfoxide was heated at 100° C. overnight. The dark brown suspension was cooled and poured into cold water. The precipitate was collected by filtration, washed with water, and recrystallized from ethanol. After drying, 2.00 g (34%) of the title compound was obtained, mp 224-226° C.

Analysis calculated for C₂₃H₃₅N₇O₁:

C, 64.91; H, 8.29; N, 23.04.

Found: C, 64.54; H, 8.22; N, 23.14.

### EXAMPLE 11

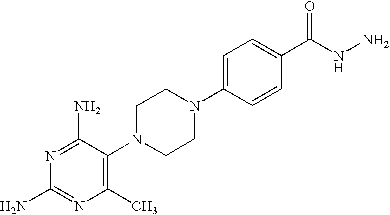

4-[4-(2,4-Diamino-6-methyl-5-pyrinidinyl)-1-piperazinyl]benzoic acid, hydrazide

A suspension of 5.0 g (0.0140 mol) of 4-[4-(2,4-diamino-6-methyl-5-pyrimidinyl)-1-piperazinyl]benzoic acid, ethyl ester in 34 mL (0.7 mol) of hydrazine hydrate was heated under reflux in a nitrogen atmosphere for about 18 hours. An additional 34 mL of hydrazine hydrate was added, and the reaction continued for an additional 24 hours. The reaction was permitted to cool to room temperature and then poured into ice water. The white precipitate was collected, washed with water, and dried to give 4.45 g (93%) of the title compound, mp 258-260° C.

Analysis calculated for C₁₆H₂₂N₈O:

C, 56.12; H, 6.48; N, 32.73.

Found: C, 56.16; H, 6.54; N, 33.01.

### EXAMPLE 12

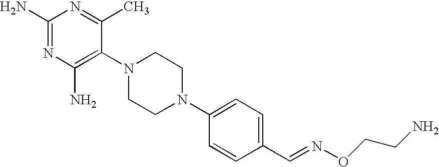

4-[4-(2,4-Diamino-6-methyl-pyrimidin-5-yl)-piperazin-1-yl]-benzaldehyde O-(2-amino-ethyl)-oxime, dihydrochloride

A suspension of 2.6 g (0.008 mol) of 4-[4-(2,4-diamino-6-methyl-pyrimidin-5-yl)-piperazin-1-yl)-benzaldehyde and 1.96 g (0.0132 mol) of H₂NO(CH₂)₂NH₂·2HCl in 200 mL of 95% ethanol was heated under reflux for 1 hour. During heating, the suspension became a yellow solution before depositing a white precipitate. The reaction was allowed to cool to room temperature, filtered, and the recovered solid was dried in vacuo to afford 2.3 g (63%) of the title compound, mp >250° C.

Analysis calculated for C₁₈H₂₆N₈O·1.60 HCl·0.30 H₂O:

C, 49.79; H, 6.55; N, 25.81; Cl, 13.06.

Found: C, 50.12; H, 6.76; N, 25.12; Cl—, 13.02.

### EXAMPLE 13

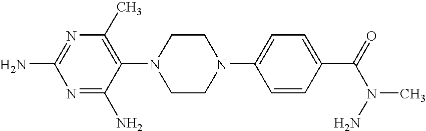

4-[4-(2,4-Diamino-6-methyl-5-pryimidinyl)-1-piperazinyl]benzoic acid, 1-methylhydrazide

To a suspension of 1.0 g (0.00305 mol) of dry 4-[4-(2,4-diamino-6-methyl-5-pryimidinyl)-1-piperazinyl]benzoic acid in 50 mL of dry dimethyl formamide (DMF) was added 3.70 mL (0.0244 mol) of freshly distilled diethylcyanophosphonate and 3.4 mL (0.0244 mol) of triethylamine. The suspension was heated to 80° C. for 15 minutes. Another 20 mL of DMF was added to aide solubility and the reaction continued at 80° C. for another 10 minutes. The resulting yellow solution was cooled to 5° C., and 1.62 mL (0.0305 mol) of methylhydrazine was added in a single portion to the suspension that formed. After stirring at 5° C. for 5 minutes, the reaction continued at room temperature for another 18 hours. The reaction was filtered, and the white solid obtained was dried to give 0.62 g of crude product. Recrystallization from 50 mL of ethanol afforded 0.30 g (27%) of the title compound. Further purification by flash chromatography [silica gel; eluting with dichloromethane/methanol (3:1)] gave 0.15 g (14%) of the title compound as a white solid, mp >275° C.

Analysis calculated for C₁₇H₂₄N₈O·0.5 H₂O:

C, 55.87; H, 6.90; N, 30.67

Found: C, 56.14; H, 6.96; N, 30.47.

### EXAMPLE 14

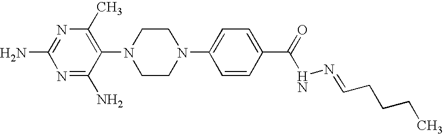

4-[4-(2,4-Diamino-6-methyl-pyrimidin-5-yl)- piperazin-1-yl]-benzoic acid 2-pentylidenehydrazide, hemiacetate

To a suspension of 0.34 g (0.001 mol) of 4-[4-(2,4-diamino-6-methyl-5-pyrimidinyl)-1-piperazinyl]benzoic acid, hydrazide and 0.13 mL (0.0012 mol) of valeraldehyde in 35 mL of ethanol was added 3.5 mL of acetic acid. The suspension was heated to reflux, and another 5.5 mL of acetic acid was added portionwise to give a solution. After refluxing for 2 hours, the reaction was cooled and filtered to give 0.33 g of a whitish solid. Recrystallization from 50 mL of boiling methanol gave 0.17 g (39%) of the title compound, mp 257-261° C.

Analysis calculated for (C₂₁H₃₀N₈O·0.5 HOAc):

C, 59.98; H, 7.32; N, 25.44.

Found: C, 59.79; H, 7.54; N, 25.33.

### EXAMPLE 15

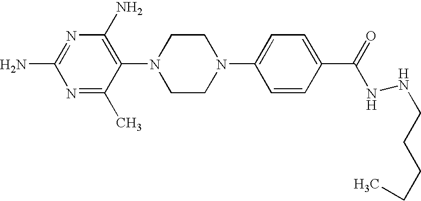

4-[4-(2,4-Diamino-6-methyl-pryimidin-5-yl)-piperazin-1-yl]-benzoic acid, N′-pentyl-hydrazide

A suspension of 1.76 g (0.00374 mol) of 4-[4-(2,4-diamino-6-methyl-pyrimidin-5-yl)-piperazin-1-yl]-benzoic acid, 2-pentylidene hydrazide (Example 14) in 300 mL of methanol was heated to reflux, and a trace of methyl orange indicator was added to give a yellow solution (pH 3.1-4.4/red-yellow). The reaction was allowed to cool slowly to room temperature, and 0.24 g (0.00374 mol) of sodium cyanoborohydride was added followed by several drops of a dry solution of hydrochloric acid in isopropanol to give the first sign of a red color. During the next 15 minutes, the HCl/iPrOH solution was added as needed until the red color persisted. The red solution was then stirred at room temperature for 1 hour. The reaction was concentrated in vacuo, and the residue was suspended in water. To the pink suspension was added concentrated NH₄OH until the reaction was basic. The light cream colored solid was collected and dried to give 1.34 g of the crude product. The solid dissolved in a minimal amount of warm methanol and flash chromatographed on silica gel eluting with 20% methanol in dichloromethane. The appropriate fractions were pooled and concentrated to give 1.0 g of a white solid which was triturated with hot ethanol to give, after drying, 0.80 g of a white solid which was redissolved in 20 mL of hot methanol. To this solution was added 50 mL of 0.1N NaOH to produce a white precipitate. The precipitate was collected and recrystallized from methanol to afford 0.32 g (21%) of the title compound as a slightly yellow solid, mp 238-242° C.

Analysis calculated for C₂₁H₃₂N₈O·0.05 HCl:

C, 60.87; H, 7.80; N, 27.04; Cl—, 0.43.

Found: C, 60.53; H, 8.01; N, 26.79; Cl, 0.45.

### EXAMPLE 16

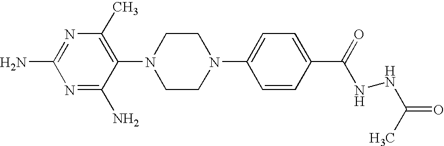

4-[4-(2,4-Diamino-6-methyl-5-pryimidinyl)-1-piperazinyl]benzoic acid, 2-acetylhydrazide, hydrochloride

To a suspension of 0.50 g (0.00146 mol) of 4-[4-(2,4-diamino-6-methyl-5-pyrimidinyl)-1-piperazinyl]benzoic acid, hydrazide in 10 mL of acetic acid was added 0.12 mL (0.00175 mol) of acetyl chloride. A heavy white precipitate formed, then disappeared leaving a gum. After stirring overnight at room temperature, a heavy white precipitate had again formed. Ether was added to the reaction, and the solid obtained after filtration was recrystallized from 10 mL of water to give 0.5 g (82%) of the title compound, mp >250° C.

Analysis calculated for C₁₈H₂₄N₈O₂·1.0 HCl:

C, 51.37; H, 5.99; N, 26.62; Cl—, 8.42.

Found: C, 51.27; H, 5.96; N, 36.9; Cl—, 8.08.

### EXAMPLE 17

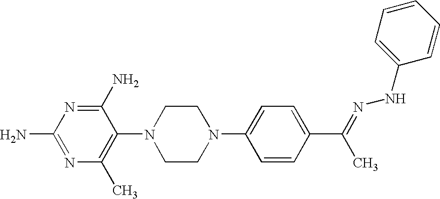

6-Methyl-5-(4-{4-[1-(phenyl-hydrazono)-ethyl]-phenyl}-piperazin-1-yl)-pyrimidine-2,4-diamine

A suspension of 3.26 g (0.01 mol) of 1-{4-[4-(2,4-diamino-6-methyl-pyrimidin-5-yl)-piperazin-1-yl]-phenyl}-ethanone in 25 mL of acetic acid and 125 mL of ethanol was heated briefly until all the solid dissolved. To the warm solution was added 2.95 mL (0.03 mol) of phenylhydrazine. The reaction stirred at room temperature for 3 hours. The cream colored precipitate was collected and recrystallized from a mixture of 200 mL of ethanol and 10 mL of acetic acid to give 3.42 g (70%) of the title compound as an off-white solid, mp 239-241° C.

Analysis calculated for C₂₃H₂₈N₈·1.25 HOAc (Acetic acid):

C, 62.30; H, 6.77; N, 22.8.

Found: C, 62.18; H, 6.74; N, 23.07.

### EXAMPLES 18-27

The compounds illustrated in Examples 18-27 were prepared by following the general procedures of the foregoing examples.

### EXAMPLE 18

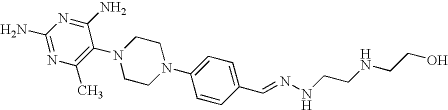

Benzaldehyde, 4-[4-(2,4-diamino-6-methyl-5-pyrimidinyl)-1-piperazinyl]-,[2-[(2-hydroxyethyl)amino]ethyl]hydrazone, methanesulfonate

Analysis calculated for C₂₀H₃₁N₉O·2.0 CH₃SO₃H·1.39 H₂O:

C, 41.89; H, 6.68; N, 19.98; S, 10.17; H₂O, 3.97.

Found: C, 41.65; H, 6.26; N, 19.89; S, 9.77; H₂O, 4.05.

### EXAMPLE 19

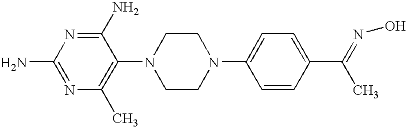

Ethanone, 1-[4-(2,4-diamino-6-methyl-5-pyrimidinyl)-1-piperazinyl]phenyl]-, oxime, monohydrochloride, mp >250° C.

### EXAMPLE 20

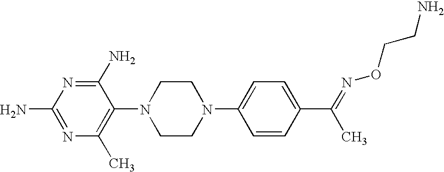

1-{4-[4-(2,4-Diamino-6-methyl-pyrimidin-5-yl)-piperazin-1-yl]-phenyl}-ethanone O-(2-amino-ethyl)-oxime, hydrochloride, mp 275° C.

Analysis calculated for C₁₉H₂₈N₈O·1.7 HCl·0.3 H₂O:

C, 50.50; H, 6.76; N, 24.80; Cl, 13.34.

Found: C, 50.12; H, 6.67; N, 25.12; Cl, 13.02.

### EXAMPLE 21

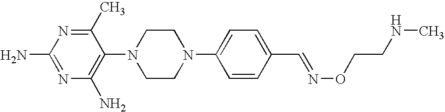

4-[4-(2,4-Diamino-6-methyl-pyrimidin-5-yl)-piperazin-1-yl]-benzaldehyde O-(2-methylamino-ethyl)-oxime, dihydrochloride, mp 260-262° C.

Analysis calculated for C₁₉H₂₈N₈O·2.0 HCl H₂O:

C, 48.00; H, 6.78; N, 23.57; Cl, 14.91; H₂O, 3.79.

Found: C, 48.09; H, 6.67; N, 23.71; Cl, 14.78; H₂O, 3.89.

### EXAMPLE 22

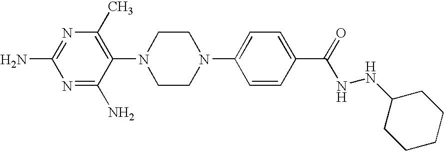

4-[4-(2,4-Diamino-6-methyl-pyrimidin-5-yl)-piperazin-1-yl]-benzoic acid, 2-cyclohexylhydrazide, mp 246-249° C.

Analysis calculated for C₂₂H₃₂N₈O·0.5 H₂O:

C, 60.94; H, 7.67; N, 25.85.

Found: C, 60.92; H, 7.52; N, 26.11.

### EXAMPLE 23

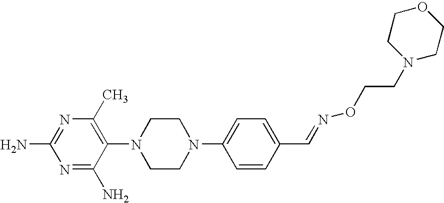

4-[4-(2,4-Diamino-6-methyl-pyrimidin-5-yl)-piperazin-1-yl]-benzaldehyde O-(2-morpholin-4-yl-ethyl)-oxime, dihydrochloride, mp 275-277° C.

Analysis calculated for C₂₂H₃₂N₈O₂·1.9 HCl·1.4 H₂O:

C, 49.39; H, 6.91; N, 20.94; Cl, 12.59; H₂O, 4.71.

Found: C, 49.27; H, 6,98; N, 20.92; Cl, 12.56; H₂O, 4.48.

### EXAMPLE 24

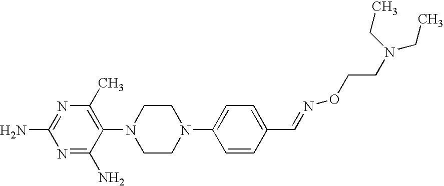

4-[4-(2,4-Diamino-6-methyl-pyrimidin-5-yl)-piperazin-1-yl]-benzaldehyde O-(2-diethylamino-ethyl)-oxime, dihydrochloride, mp 283-284° C.

Analysis calculated for C₂₂H₃₄N₈O·2 HCl:

C, 51.42; H, 7.43; N, 21.81; Cl, 13.11;H₂O, 3.51.

Found: C, 51.49; H, 7.35; N, 21.65; Cl, 13.17; H₂O, 3.21.

### EXAMPLE 25

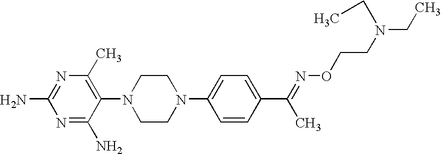

1-{4-[4-(2,4-Diamino-6-methyl-pyrimidin-5-yl)-piperazin-1-yl]-phenyl}-ethanone O-(2-diethylamino-ethyl)-oxime, dihydrochloride, mp 234-236° C.

Analysis calculated for C₂₃H₃₆N₈O·2.85HCl·1.7 H₂O:

C, 48.03; H, 7.40; N, 19.48; Cl, 17.57; H₂O, 5.21.

Found: C, 47.72; H, 7.33; N, 19.47; Cl, 17.24; H₂O, 4.85.

### EXAMPLE 26

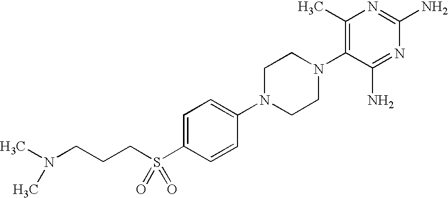

5-{4-[4-(3-Dimethylamino-propane-1-sulfonyl)-phenyl]-piperazin-1-yl}-6-methyl-pyrimidine-2,4-diamine, mp 315-318° C.

Analysis calculated for C₂₀H₃₁N₇O₂S·2.0 HCl·1.6 H₂O:

C, 44.87; H, 6.82; H, 18.32; Cl, 13.25; H₂O, 5.38.

Found: C, 45.08; H, 6.64; N, 18.24; Cl, 13.01; H₂O, 5.56.

### EXAMPLE 27

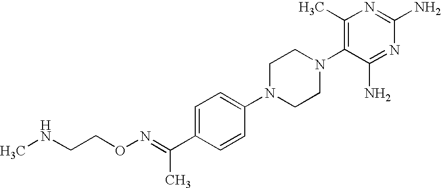

1-{4-[4-(2,4-Diamino-6-methyl-5-pyrirnidinyl)-piperazin-1-yl]-phenyl}-ethanone O-(2-methylamino-ethyl)-oxime, trihydrochloride, mp 217-219° C.

Analysis calculated for C₂₀H₃₀N₈O·3 HCl·2.6 H₂O:

C, 43.30; H, 6.94; N, 20.20; Cl, 19.17; H₂O, 8.44.

Found: C, 43.32; H, 6.90; N, 20.25; Cl, 19.14; H₂O, 8.60.

### BIOLOGICAL EXAMPLES

Activity of Compounds of the Present Invention Against P-Glycoprotein Negative Cell Lines

The compounds of the present invention comprise a series of diaminopyrimidines that, like TMQ, inhibit dihydrofolate reductase (see Table 1 for selected examples) and that overcome P-glycoprotein-induced MDR in a variety of P-glycoprotein positive tumors both in vitro and in vivo (see Table 2 for selected examples). In contrast to TMQ, these compounds are as active in vitro against P-glycoprotein positive P388/ADR leukemia cells as they are in the parent P388S cells that do not express P-glycoprotein.

TABLE 1

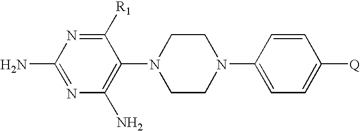

| Compound | R₁ | Q | Kᵢ (pM) |
|---|---|---|---|
| A* | CH₃— | Glutamate | 53 |
| B | CH₃— | —COOH | 201 |
| C | CH₃CH₂ | Glutamate (OCH₃CH₃) | 109 |
| D | CH₃— | —COCH₃ | 131 |
| E | CH₃— | —SO₂CH₃ | 138 |
| Example 1 | CH₃— | —CHO | 73 |
| Methotrexate | | | 3 |
| Trimetrexate | | | 43 |

*A-E represent the following compounds:
A) {2-[4-[4[(2,4-Diamino-6-methyl-pyrimidin-5-yl)-piperazin-1-yl]-benzylamino}-pentanedioic acid;
B) 4-[4-(2,4-Diamino-6-methyl-pyrimidin-5-yl)-piperzain-1-yl]-benzoic acid;
C) 2-{4-[4-(2,4-Diamino-6-ethyl-pyrimidin-5-yl)-pierazin-1-yl]-benzoylamino}pentanedioic acid diethyl ester;
D) 1-{4-[4-(2,4-Diamino-6-methyl-pyrimidin-5-yl)-piperazin-1-yl]-phenyl}-ethanone; and,
E) 4-[4-(4-Methanesulfonyl-phenyl)-piperzin-1-yl]-6-methyl-pyrimidine-2,4-diamine.

To test the compounds of the present invention in vitro, the following methodology was used. P388S and P388/ADR cells were cultured in RPMI-1640 media supplemented with 10% horse serum and 10 μM 2-mercaptoethanol. HT29 cells were cultured in Dulbecco's MEM (DMEM) media supplemented with 10% dialyzed fetal calf serum. C26 cells were cultured in DMEM/F12 media supplemented with 10% dialyzed fetal calf serum. To examine the potential synergy between antifolates and FU plus LV, cells were exposed to TMQ or the diaminopyrimidines for a total of 24 hours. LV was added for the final 4.5 hours, and FU was added for the final 4 hours of the 24-hour incubation period. Cells were then washed three times in drug-free media and incubated for 2 to 4 days in drug free media. The effect of antifolates ±LV and FU was determined by sulphorhodamine B method as previously described (Skehan P L, Storeng R., Scudiero D., et al., Evaluation of colorimetric protein and biomass stains for assaying in vitro drug effects upon human tumor cell lines. Proc. Am. Assoc. Cancer Res., 1989;30:612). The results are shown in Table 3.

To test compounds of the present invention in vivo, the following methodology was used. P388 leukemia and its Adriamycin resistant subline, P388/ADR were passed weekly in DBA/2 mice as i.p. implants of 10⁵ and 10⁶ cells, respectively. Animals bearing P388/ADR passage were treated with 7 mg/kg Adriamycin on Day 3 post-implant as a resistance selection pressure. C26/clone 10 was passed every 2 weeks into Balb/c mice as tumor fragments (approximately 30 mg) which were obtained from tumors 1 g in mass. The results are shown in Table 2.

Anticancer activity was evaluated in CD2F₁ mice (Balb/c×DBA/2). P388 parent and resistant lines were compared in identical tests conducted simultaneously. Test mice weighing 18-22 g were randomized and implanted with 10⁵ P388 or P388/ADR leukemia cells on Day 0. Animals were treated i.p., Days 3-11, on the basis of average cage weight. Animals in each experiment were held for 60 days. Adriamycin treated groups (i.p. on Days 3, 7, and 11) were included in each test to verify resistance of the P88/ADR line to Adriamycin. Test compounds were dissolved in 0.5% sodium bicarbonate (final pH=10), PGIMS (compound dissolved in 10% methane sulfonic acid equivalent to 10% of the final volume and diluted to volume with 10% proplylene glycol in water, final pH=4.0), or suspended in 0.5% methylcellulose in water. Adriamycin was dissolved in saline. In each test a dose response ranging from toxic to ineffective dose levels was evaluated for each compound so that an indication of the therapeutic index could be obtained. The table values are data obtained at the maximum tolerated dose: the highest dose level in the does response producing less than 10% lethality (<LD10). Calculation of the median day of death and percent T/C were performed as described previously (1, 2, 3). Animals surviving more than 60 days were excluded from the calculation of % T/C. Percent T/C values in excess of 130 indicate an antitumor effect in the P388 and P388/ADR model systems. The results are shown in Table 2.

Colon 26/clone 10 (C26/10) life span studies were conducted in CD2F₁ mice. C26/C10 cells were implanted i.p. on Day 0. Animals were treated i.p. with test agents in the vehicles described above on Days 3-7 and 10-14. Adriamycin was dosed i.p. in saline on Days 5, 9, and 13. Calculations of the median day of death and % T/C were as described previously with animals surviving more than 60 days excluded from the calculations (Schabel et al., Quantitative evaluation of anti-cancer agent activity in experimental animals. Pharmacol. Ther., 1977;1:411-435; Leopold et al., Anthrapyrazoles, a new class of intercalating agents with high-level broad spectrum activity against murine tumors. Cancer Res., 1985;45:5532-5539; Elliot et al., Sequence and schedule dependent synergy of trimetrexate in combination with 5 fluorouracil in vitro and in mice. Cancer Res., 1984;49:5586-5640). Percent T/C values in excess of 130 are indicative of an antitumor effect in this model. The results are shown in Table 2.

For the tumor growth delay studies with C26/10, CD2F₁ mice were implanted s.c. with 30 mg tumor fragments in the region of the right axilla with the aid of a trocar on Day 0. Animals were treated i.p. with test agents in the vehicles described above on Days 3-7 and 10-14. Adriamycin was dosed i.p. in saline on Days 5, 9, and 13. Tumors in the treated and control groups were measured twice weekly in two perpendicular dimensions and tumor mass calculated according to the formula: mass (mg)=(a×b²)/2 where “b” is the smallest tumor dimension and “a” is the largest. Calculations of tumor growth delay, defined as the median time (in days) for the treated tumors to reach 750 mg in size minus the median time (in days) for the control tumors to reach 750 mg in size, were as described previously (Schabel, 1977; Leopold, 1985; Elliot, 1984). Growth delays in excess of 5 to 6 days in the C26/10 model are considered to be an antitumor effect. The results are shown in Table 2.

Against P388/ADR ascites tumors in vivo, the activity of the diaminopyrimidines shown in Table 2 was similar to that observed against P388S (Table 2). Anticancer agents such as TMQ or Adriamycin, which are substrates for P-glycoprotein, were inactive against P388/ADR tumors in vivo. Several of the diaminopyrimidine antifolates were also tested against murine Colon 26 (C26) tumor in vivo (Table 2). C26 expresses P-glycoprotein, but not to nearly the extent as P388/ADR cells, and in this regard may be more clinically relevant to the levels of P-glycoprotein expressed in human colon cancer. Several diaminopyrimidines demonstrated significant activity against in vivo C26 colon tumors (Table 2). Example 1 was actually curative against this P-glycoprotein positive colon tumor. In contrast, TMQ was without activity against C26 (T/C×100>130 represents in vivo antitumor activity). The results are shown in Table 2.

TABLE 2
Activity of Antifolates and Adriamycin on In Vitro and
In Vivo Tumor Models

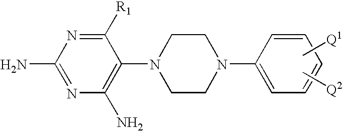

| | | | In vitro Studies | | | In vivo Studies | | | |
|---|---|---|---|---|---|---|---|---|---|
| | | | P388S | P388/ | | | P388/ | | |
| | | | IC₅₀ | ADR IC₅₀ | | P388S | ADR | C26 ip | C26 sc |
| Compound | R₁ | Q₁, Q₂ | (ng/mL) | (ng/mL) | R/S | T/C | T/C | T/C | T-C |
| Trimetrexate | | | 6.9 | 152 | 22 | 164 | 113 | 119 | 6.8 |
| Adriamycin | | | 75 | 3900 | 52 | 250 | 108 | 180 | NT |
| D* | Me | COCH₃ | 10 | 10 | 1 | 182 | 180 | 171 | 17.3 |
| Ex. 1 | Me | CHO | 5 | 6.8 | 1.1 | 190 | 169 | 263 | 7.6 |
| F | Me | SO₃H | 33 | 40 | 1.1 | 260 | NT | NT | NT |
| G | H | COOH | 76 | 74 | 0.98 | 220 | 245 | 202 | 4.9 |
| H | Me | 4Ac, 3F | 2.6 | 29 | 1.1 | 170 | NT | 169 | 9.4 |
| Ex. 11 | Me | 4-CONH NH₂ | 10.5 | 16.1 | 1.5 | 195 | 161 | 164 | NT |
| Ex. 12 | Me | CNOCH₂ | 11 | 11.7 | 1.1 | NT | NT | NT | 5.2 |
| | | CH₂NH₂ | | | | | | | |

*D, F, G, and H represent the following compounds:
D) 1-{4-[4-(2,4-Diamino-6-methyl-pyrimidin-5-yl)-piperazin-1-yl]-phenyl}-ethanone;
F) 4-[4-(2,4-Diamino-6-methyl-pyrimidin-5-yl)-pyrimidin-5-yl]-benzoic acid;
G) 4-[4[(2,4-Diamino-pyrimidin-5-yl)-piperazin-1-yl]-benzoic acid; and
H) 1-{4-[4-(2,4-Diamino-6-methyl-pyrimidin-5-yl)-piperazin-1-yl]-2-fluoro-phenyl}-ethanone.

Several diaminopyrimidines as well as trimetrexate were tested in vitro against HT29 human colon adenocarcinoma cells (Table 3). These cells do not express P-glycoprotein. All of the antifolates including TMQ appeared to have similar effects on HT29 cell growth. The results are shown in Table 3. Also, all of the compounds showed a synergistic pattern of activity with FU and LV. These results are similar to those reported for TMQ combined with FU/LV against a P-glycoprotein negative human leukemia cell line, CCRF-CEM (Romanini A., Li W. W., Colofiore J. R., Bertino J. R., Leucovorin enhances cytotoxicity of trimetrexate/fluorouracil, but not methotrexate/fluorouracil, in CCRF-CEM cells. J. Natl. Cancer Inst., 1992;84:1033-1038).

TABLE 3
Effect of Combination of Antifolates With 5-FU (FU) and Leucovorin
(LV) on HT-29 Proliferation

| | | | | | Antifol + |
|---|---|---|---|---|---|
| Compound | FU | Antifol | Antifol + LV | Antifol + FU | LV + FU |
| Trimetrexate | 73* | 75 | 77 | 35 | 30 |
| D^{@} | 73 | 92 | 90 | 50 | 42 |
| H | 73 | 74 | 77 | 52 | 40 |
| Example 11 | 73 | 87 | 80 | 42 | 33 |
| Example 12 | 73 | 52 | 67 | 35 | 27 |

*Numerical values indicate percent of control following exposure of cells to antifolate for 24 hours. LV (10 μM) was added for the final 4.5 hours, and 5 μM FU was added for the final 4 hours of the 24-hour incubation. Antifolate concentration was 62.5 nM.
D^{@} and H represent the following compounds:
D) 1-{4-[4-(2,4-Diamino-6-methyl-pyrimidin-5-yl)-piperazin-1-yl]-phenyl}-ethanone; and
H) 1-{4-[4-(2,4-Diamino-6-methyl-pyrimidin-5-yl)-piperazin-1-yl]-2-fluoro-phenyl}-ethanone.

Several antifolates including TMQ were also tested against P388 murine leukemia cells in vitro (Table 4). These leukemic cells also do not appear to express P-glycoprotein. All of the compounds tested showed significant synergy with FU and LV.

TABLE 4
Effect of Combination of Antifolates with 5-FU (FU) and Leucovorin
(LV) on P388 Proliferation

| | | | | | Antifol + |
|---|---|---|---|---|---|
| Compound | FU | Antifol | Antifol + LV | Antifol + FU | LV + FU |
| Trimetrexate | 90* | 80 | 85 | 12 | 9 |
| D^{@} | 90 | 70 | 110 | 10 | 5 |
| H | 90 | 82 | 86 | 20 | 15 |
| Example 12 | 90 | 100 | 97 | 20 | 16 |
| Example 13 | 90 | 85 | 85 | 10 | 7 |

*Numerical values indicate percent of control following exposure of cells to antifolate for 24 hours. LV (10 μM) was added for the final 4.5 hours, and 5 μM FU was added for the final 4 hours of the 24-hour incubation. Antifolate concentration was 1.0 nM.
D^{@} and H represent the following compounds:
D) 1-{4-[4-(2,4-Diamino-6-methyl-pyrimidin-5-yl)-piperazin-1-yl]-phenyl}-ethanone; and
H) 1-{4-[4-(2,4-Diamino-6-methyl-pyrimidin-5-yl)-piperazin-1-yl]-2-fluoro-phenyl}-ethanone.

However, when tested against P-glycoprotein positive P388/ADR cells in vitro, TMQ at a concentration 10-fold higher than the other antifolates, had little activity alone nor was a significant increase in activity observed by the addition of either FU or LV (Table 5).

TABLE 5
Effect of Combination of Antifolates with 5-FU (FU) and Leucovorin
(LV) on P388/ADR Proliferation

| | | | | | Antifol + |
|---|---|---|---|---|---|
| Compound | FU | Antifol | Antifol + LV | Antifol + FU | LV + FU |
| Trimetrexate | 90* | 92 | 100 | 83 | 76 |
| B** | 90 | 62 | 70 | 30 | 28 |
| C** | 90 | 88 | 80 | 40 | 30 |
| Example 11 | 90 | 77 | 80 | 48 | 16 |
| Example 12 | 90 | 65 | 65 | 30 | 25 |

*Numerical values indicate percent of control following exposure of cells to antifolate for 24 hours. LV (10 μM) was added for the final 4.5 hours, and 5 μM FU was added for the final 4 hours of the 24-hour incubation. TMQ concentration was 10 nM. Other antifolate concentration was 1 nM.
**B and C represent the following compounds:
B) 4-[4-(2,4-Diamino-6-methyl-pyrimidin-5-yl)-piperzain-1-yl]-benzoic acid; and
C) 2-{4-[4-(2,4-Diamino-6-ethyl-pryimidin-5-yl)-pierazin-1-yl]-benzoylamino}pentanedioic acid diethyl ester.

The four diaminopyrimidines, on the other hand, at 10-fold lower concentration, all had significant synergistic activity when combined with FU and LV. Similar findings were observed with C26 murine colon tumor cells (Table 6). C26 cells have a level of P-glycoprotein expression similar to many human colon tumor biopsies. At 0.125 μM, TMQ had no effect on C26 proliferation alone and there was no significant synergy observed if either FU and/or LV were added to the media. However, both PD 115748 and PD 132625 demonstrated significant synergistic activity when combined with FU or FU/LV (Table 6).

TABLE 6
Effect of Combination of Antifolates With 5-(FU) and Leucovorin (LV)
on C26 Proliferation

| | Percent of Control^{a} | | | | |
|---|---|---|---|---|---|
| | | | | | Antifol + FU + |
| Compound | FU | Antifol | Antifol + LV | Antifol + FU | LV |
| Trimetrexate | 72 | 101 | 98 | 65 | 64 |
| PD 115748 | 72 | 60 | 65 | 35 | 30 |
| PD 132625 | 72 | 72 | 75 | 30 | 21 |

aCells were exposed to antifolate for 24 hours. LV (10 μM) was added for the final 4.5 hours, and 5 μM FU was added for the final 4 hours of this 24-hour incubation. Antifolate concentration was 0.125 μM.

What is claimed is:

1. A method of treating cancer, the method comprising administering to a patient having cancer, a therapeutically effective amount of a combination of:
1) 5-fluorouracil;
2) leucovorin; and
3) a compound of Formula I 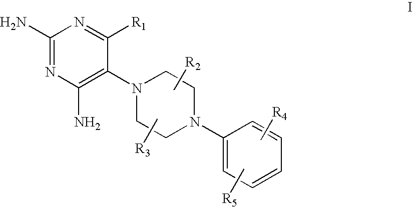
wherein
R₁ is hydrogen or alkyl of from one to six carbon atoms;
R₂ and R₃ are independently hydrogen or methyl;
R₄ and R₅ are independently:
hydrogen,
halogen,
nitro,
cyano,
trifluoromethyl,
hydroxyl,
alkyl of from one to six carbon atoms,
alkoxyl of from one to six carbon atoms,
alkanoyl of from one to six carbon atoms;
—NR₆R₇, where R₆ and R₇ are independently
hydrogen,
alkyl of from one to six carbon atoms,
alkanoyl of from one to six carbon atoms;
—COOR₈ where R₈ is
hydrogen,
a pharmaceutically acceptable metal cation,
a pharmaceutically acceptable amine cation,
alkyl of from one to six carbon atoms;
—CONR₉ R₁₀ where R₉ and R₁₀ are independently
hydrogen,
alkyl of from one to six carbon atoms,
alkyl of from one to six carbon atoms, substituted with one or two carboxyl groups,
alkyl of from one to six carbon atoms, substituted with one or two carboxyl groups and one —OH, —SH, or —NH₂ group,
alkyl of from one to six carbon atoms, substituted with one or two carboalkoxy groups of from one to six carbon atoms,
alkyl of from one to six carbon atoms, substituted with one or two carboalkoxy groups of from one to six carbon atoms and one —OH, —SH, or —NH₂ group; 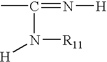
where R₁₁ is hydrogen, or alkyl of from one to six carbon atoms; or
—SO₂R₁₂ where R₁₂ is
hydroxyl,
alkyl of from one to six carbon atoms,
alkoxy of from one to six carbon atoms, or
—NR₁₃R₁₄ where R₁₃ and R₁₄ are independently hydrogen or alkyl of from one to six carbon atoms;
and the pharmaceutically acceptable salts thereof.

2. A method of treating cancer, the method comprising administering to a patient having cancer, a therapeutically effective amount of a combination of:
1) a thymidylate synthase inhibitor; and
2) a compound of Formula I 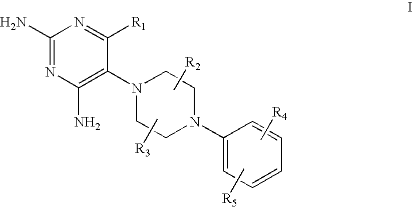
wherein
R₁ is hydrogen or alkyl of from one to six carbon atoms;
R₂ and R₃ are independently hydrogen or methyl;
R₄ and R₅ are independently:
hydrogen,
halogen,
nitro,
cyano,
trifluoromethyl,
hydroxyl,
alkyl of from one to six carbon atoms,
alkoxyl of from one to six carbon atoms,
alkanoyl of from one to six carbon atoms;
—NR₆R₇, where R₆ and R₇ are independently
hydrogen,
alkyl of from one to six carbon atoms,
alkanoyl of from one to six carbon atoms;
—COOR₈ where R₈ is
hydrogen,
a pharmaceutically acceptable metal cation,
a pharmaceutically acceptable anine cation,
alkyl of from one to six carbon atoms;
—CONR₉ R₁₀ where R₉ and R₁₀ are independently
hydrogen,
palkyl of from one to six carbon atoms,
alkyl of from one to six carbon atoms, substituted with one or two carboxyl groups,
alkyl of from one to six carbon atoms, substituted with one or two carboxyl groups and one —OH, —SH, or —NH₂ group,
alkyl of from one to six carbon atoms, substituted with one or two carboalkoxy groups of from one to six carbon atoms,
alkyl of from one to six carbon atoms, substituted with one or two carboalkoxy groups of from one to six carbon atoms and one —OH, —SH, or —NH₂ group; 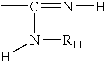
where R₁₁ is hydrogen, or alkyl of from one to six carbon atoms; or
—SO₂R₁₂ where R₁₂ is
hydroxyl,
alkyl of from one to six carbon atoms,
alkoxy of from one to six carbon atoms, or
—NR₁₃R₁₄ where R₁₃ and R₁₄ are independently hydrogen or alkyl of from one to six carbon atoms;
and the pharmaceutically acceptable salts thereof.

3. The method of claim 1 wherein the cancer is colorectal cancer.

4. The method of claim 2 wherein the cancer is colorectal cancer.

5. The method of claim 2 wherein the thymidylate synthase inhibitor is 2-({5-[Methyl-(2-methyl-4-oxo-3,4-dihydro-quinazolin-6-ylmethyl)-amino]-thiophene-2-carbonyl}-amino)-pentanedioic acid.

6. The method of claim 2 wherein the thymidylate synthase inhibitor is:
2-{4-[2-(2-Amino-4-oxo-4,7-dihydro-3H-pyrrolo[2,3-d]pyrimidin-5-yl)-ethyl]-benzoylamino}-pentanedioic acid;
N⁶-Methyl-N⁶-[4-(morpholine-4-sulfonyl)-benzyl]-benzo[cd]indole-2,6-diamine; compound with 3,4,5,6-tetrahydroxy-tetrahydro-pyran-2-carboxylic acid; or
2-Amino-6-methyl-5-(pyridin-4-ylsulfanyl)-3H-quinazolin-4-one.

7. The method of claim 1 wherein the compound of Formula I is:
4-[4-(2,4-Diamino-6-methyl-5-pyrimidinyl)-1-piperazinyl]benzoic acid;
4-[4-(2,4-Diamino-6-methyl-5-pyrimidinyl)-1-piperazinyl]benzoic acid ethyl ester;
5-[4-(4-Cyanophenyl)-1-piperazinyl]-6-methyl-2,4-pyrimidinediamine;
N-[4-[4-(2,4-Diamino-6-methyl-5-pyrimidinyl)- 1 -piperazinyl]-benzoyl]-L-glutamic acid;
N-[4-[4-(2,4-Diamino-6-methyl-5-pyrimidinyl)-1-piperazinyl]-benzoyl]-L-glutamic acid diethyl ester;
6-Methyl-5-[4-(4-nitrophenyl)-1-piperazinyl]-2,4-pyrimidinediamine;
5-[4-(4-Acetylphenyl)-1-piperazinyl]-6-methyl-2,4-pyrimidinediamine;
4-[4-(2,4-Diamino-6-methyl-pyrimidinyl)-1-piperazinyl]-N-methyl benzamide;
[4-(2,4-Diamino-6-methyl-5-pyrimidinyl)-1-piperazinyl]benzoyl]glycine;
N-[4-[4-(2,4-Diamino-6-methyl-5-pyrimidinyl)-1-piperazinyl]benzoyl]glycine methyl ester;
6-Methyl-5-[4-[4-(methylsulfonyl)phenyl]-1-piperazinyl]-2,4-pyrimidinediamine;
5- [4-(4-Cyanophenyl)-1-piperazinyl]-6-ethyl-2,4-pyrimidinediamine;
4-[4-(2,4-Diamino-6-ethyl-5-pyrimidinyl)-1-piperazinyl]benzoic acid;
N-[4-[4-(2,4-Diamino-6-ethyl-5-pyrimidinyl)-1-piperazinyl]benzoyl]-L-glutamic acid;
N-[4-[4-(2,4-Diamino-6-ethyl-5-pyrimidinyl)-1-piperazinyl]benzoyl]-L-glutamic acid diethyl ester;
5-[4-(4-Cyanophenyl)-1-piperazinyl]-6-propyl-2,4-pyrimidinediamine;
4-[4-(2,4-Diamino-6-propyl-5-pyrimidinyl)-1-piperazinyl]benzoic acid;
N-[4-[4-(2,4-Diamino-6-propyl-5-pyrimidinyl)-1-piperazinyl]benzoyl]-L-glutamic acid;
N-[4-[4-(2,4-Diamino-6-propyl-5-pyrimidinyl)-1-piperaziny]benzoyl]-L-glutamic acid diethyl ester;
4-[4-(2,4-Diamino-6-propyl-5-pyrimidinyl)-1-piperazinyl]benzoic acid ethyl ester;
6-Methyl-5-(4-phenyl-1-piperazinyl)-2,4-pyrimidinediamine, hydrochloride;
5-[4-(4-Chlorophenyl)-1-piperazinyl]-6-methyl-2,4-pyrimidinediaminey;
5-[4-(4-Aminophenyl-1-piperazinyl]-6-methyl-2,4-pyrimidinediamine;
N-[4-[4-(2,4-Diamino-6-methyl-5-pyrimidinyl)-1-piperazinyl]benzoyl]-glycine;
4-[4-(2,4-Diamino-6-methyl-5-pyrimidinyl)-1-piperazinyl]-N,N-dimethylbenzenesulfonamide; or
6-Methyl-5-[4-[4-trifluoromethyl)phenyl]-1-piperazinyl]-2,4-pyrimidinediamine.

8. The method of claim 2 wherein the compound of Formula I is:
4-[4-(2,4-Diamino-6-methyl-5-pyrimidinyl)-1-piperazinyl]benzoic acid;
4-[4-(2,4-Diamino-6-methyl-5-pyrimidinyl)-1-piperazinyl]benzoic acid ethyl ester;
5-[4-(4-Cyanophenyl)-1-piperazinyl]-6-methyl-2,4-pyrimidinediamine;
N-[4-[4-(2,4-Diamino-6-methyl-5-pyrimidinyl)-1-piperazinyl]-benzoyl]-L-glutamic acid;
N-[4-[4-(2,4-Diamino-6-methyl-5-pyrimidinyl)-1-piperazinyl]-benzoyl]-L-glutamic acid diethyl ester;
6-Methyl-5-[4-(4-nitrophenyl)-1-piperazinyl]-2,4-pyrimidinediamine;
5-[4-(4-Acetylphenyl)-1-piperazinyl]-6-methyl-2,4-pyrimidinediamine;
4-[4-(2,4-Diamino-6-methyl-pyrimidinyl)-1-piperazinyl]-N-methyl benzamide;
[4-(2,4-Diamino-6-methyl-5-pyrimidinyl)-1-piperazinyl]benzoyl]glycine;
N-[4-[4-(2,4-Diamino-6-methyl-5-pyrimidinyl)-1-piperazinyl]benzoyl]glycine methyl ester;
6-Methyl-5-[4-[4-(methylsulfonyl)phenyl]-1-piperazinyl]-2,4-pyrimidinediamine;
5-[4-(4-Cyanophenyl)-1-piperazinyl]-6-ethyl-2,4-pyrimidinediamine;
4-[4-(2,4-Diamino-6-ethyl-5-pyrimidinyl)-1-piperazinyl]benzoic acid;
N-[4-[4-(2,4-Diamino-6-ethyl-5-pyrimidinyl)-1-piperazinyl]benzoyl]-L-glutamic acid;
N-[4-[4-(2,4-Diamino-6-ethyl-5-pyrimidinyl)-1-piperazinyl]benzoyl]-L-glutamic acid diethyl ester;
5-[4-(4-Cyanophenyl)-1-piperazinyl]-6-propyl-2,4-pyrimidinediamine;
4-[4-(2,4-Diamino-6-propyl-5-pyrimidinyl)-1-piperazinyl]benzoic acid;
N-[4-[4-(2,4-Diamino-6-propyl-5-pyrimidinyl)-1-piperazinyl]benzoyl]-L-glutamic acid;
N-[4-[4-(2,4-Diamino-6-propyl-5-pyrimidinyl)-1-piperazinyl]benzoyl]-L-glutamic acid diethyl ester;
4-[4-(2,4-Diamino-6-propyl-5-pyrirnidinyl)-1-piperazinyl]benzoic acid ethyl ester;
6-Methyl-5-(4-phenyl-1-piperazinyl)-2,4-pyrimidinediamine, hydrochloride;
5-[4-(4-Chlorophenyl)-1-piperazinyl]-6-methyl-2,4-pyrimidinediaminey;
5-[4-(4-Aminophenyl-1-piperazinyl]-6-methyl-2,4-pyrimidinediamine;
N-[4-[4-(2,4-Diamino-6-methyl-5-pyrimidinyl)-1-piperazinyl]benzoyl]-glycine;
4-[4-(2,4-Diamino-6-methyl-5-pyrimidinyl)-1-piperazinyl]-N,N-dimethylbenzenesulfonamide; or
6-Methyl-5-[4-[4-trifluoromethyl)phenyl]-1-piperazinyl]-2,4-pyrimidinediamine.

9. The method of claim 1 wherein the cancer is a P-glycoprotein positive cancer.

10. The method of claim 2 wherein the cancer is a P-glycoprotein positive cancer.

11. A compound of Formula II: 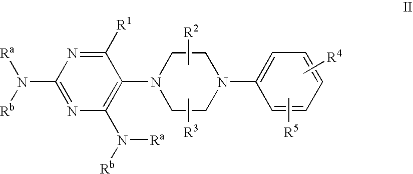
wherein
each n is independently 0 to 6;
R^{a} and R^{b} are independently hydrogen or C₁-C₆ alkyl;
R¹ is C₁-C₆ alkyl or 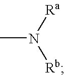
R² and R³ are independently hydrogen, —C₁-C₆ alkyl, —CH₂OH, or —OH;
R⁴ is hydrogen;
R⁵ is —(CH₂)ₙNH₂, 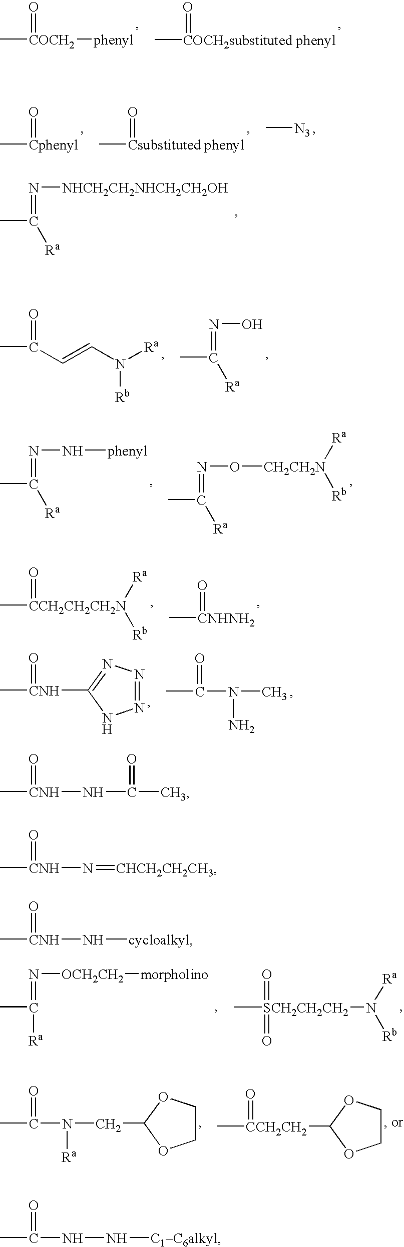
and the pharmaceutically acceptable salts thereof.

12. A compound of Formula II: 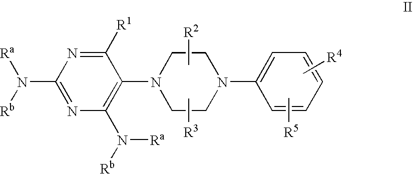
wherein
each n is independently 0 to 6;
R^{a} and R^{b} are independently hydrogen or C₁-C₆ alkyl;
R¹ is C₂-C₆ alkyl or 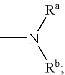
R² and R³ are independently hydrogen, —C₁-C₆ alkyl, —CH₂OH, or —OH;
R⁴ is hydrogen;
R⁵ is —(CH₂)ₙ—OH, —(CH₂)ₙNH₂, 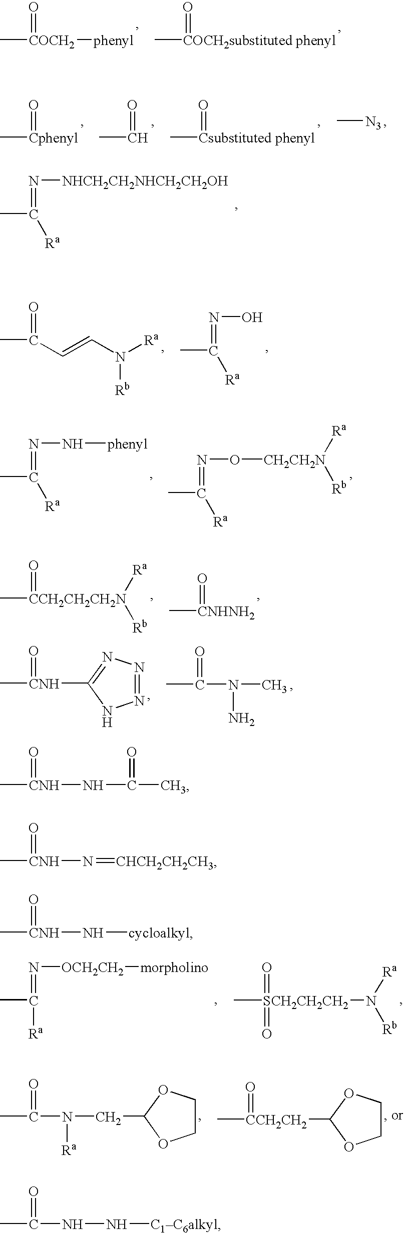
and the pharmaceutically acceptable salts thereof.

13. A compound in accordance with claim 11 or claim 12 wherein R^{a} and R^{b} are independently hydrogen or methyl.

14. A compound in accordance with claim 11 wherein R¹ is methyl.

15. A compound in accordance with claim 11 or claim 12 wherein R² and R³ are hydrogen.

16. A compound in accordance with claim 11 wherein
R^{a} and R^{b} are independently hydrogen or methyl;
R¹ is methyl;
R² and R³ are hydrogen; and
R⁴ is hydrogen.

17. The compounds:
4-[4-(2,4-Diamino-methyl-pyrimidin-5-yl)-piperazin-1-yl)-benzaldehyde;
1-{4-[4-(2,4-Diamino-6-methyl-5-pyrimidinyl)-1-piperazinyl]-phenyl}-4-(diethyl-amino)-1-butanone;
4-[4-(2,4-Diamino-6-methyl-5-pyrimidinyl)-1-piperazinyl]benzoic acid, hydrazide;
4-[4-(2,4-Diamino-6-methyl-pyrimidin-5-yl)-piperazin-1-yl]-benzaldehyde O-(2-amino-ethyl)-oxime, dihydrochloride;
4-[4-(2,4-Diamino-6-methyl-5-pryimidinyl)-1-piperazinyl]benzoic acid, 1-methylhydrazide;
4-[4-(2,4-Diamino-6-methyl-pyrimidin-5-yI)- piperazin-1-yl]-benzoic acid pentylidenehydrazide, hemiacetate;
4-[4-(2,4-Diamino-6-methyl-pryimidin-5-yl)-piperazin-1-yl]-benzoic acid, N′-pentyl-hydrazide;
4-[4-(2,4-Diamino-6-methyl-5-pryimidinyl)-1-piperazinyl]benzoic acid, 2-acetylhydrazide, hydrochloride;
6-Methyl-5-(4-{4-[1-(phenyl-hydrazono)-ethyl]-phenyl}-piperazin-1-yl)-pyrimidine-2,4-diamine;
Benzaldehyde, 4-[4-(2,4-diamino-6-methyl-5-pyrimidinyl)-1-piperazinyl]-, [2-[(2-hydroxyethyl)amino]ethyl]hydrazone, methanesulfonate;
1-{4-[4-(2,4-Diamino-6-methyl-pyrimidin-5-yl)-piperazin-1-yl]-phenyl}-ethanone O-(2-amino-ethyl)-oxime, hydrochloride;
4-[4-(2,4-Diamino-6-methyl-pyrimidin-5-yl)-piperazin-1-yl]-benzaldehyde O-(2-methylamino-ethyl)-oxime, dihydrochloride;
4-[4-(2,4-Diamino-6-methyl-pyrimidin-5-yl)-piperazin-1-yl]-benzoic acid, 2-cyclohexylhydrazide;
4-[4-(2,4-Diamino-6-methyl-pyrimidin-5-yl)-piperazin-1-yl]-benzaldehyde O-(2-morpholin-4-yl-ethyl)-oxime;
4-[4-(2,4-Diamino-6-methyl-pyrimidin-5-yl)-piperazin-1-yl]-benzaldehyde O-(2-diethylamino-ethyl)-oxime, dihydrochloride;
1-{4-[4-(2,4-Diamino-6-methyl-pyrimidin-5-yl)-piperazin-1-yl]-phenyl}-ethanone O-(2-diethylamino-ethyl)-oxime, dihydrochloride;
5-{4-[4-(3-Dimethylamino-propane-1-sulfonyl)-phenyl]-piperazin-1-yl}-6-methyl-pyrimidine-2,4-diamine; and
1-{4-[4-(2,4-Diamino-6-methyl-5-pyrimidinyl)-piperazin-1-yl]-phenyl}-ethanone O-(2-methylamino-ethyl)-oxime, trihydrochloride.

18. A pharmaceutical composition that comprises a compound of claim 11 or claim 12.

19. A compound of Formula II: 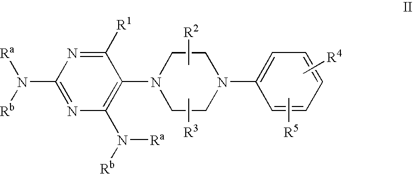
wherein
each n is independently 0 to 6;
R^{a} and R^{b} are independently hydrogen or C₁-C₆ alkyl;
R¹ is C₁-C₆ alkyl or 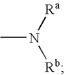
R² and R³ are independently hydrogen, —C₁-C₆ alkyl, —CH₂OH, or —OH;
R⁴ is hydrogen;
R⁵ is —(CH₂)ₙ—OH,
and the pharmaceutically acceptable salts thereof.

20. A compound of Formula III: 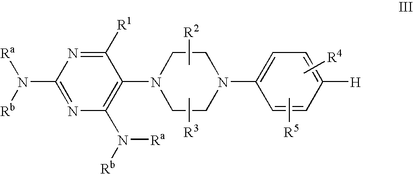
wherein
each n is independently 0 to 6;
R^{a} and R^{b} are independently hydrogen or C₁-C₆ alkyl;
R¹ is C₁-C₆ alkyl or 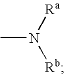
R² and R³ are independently hydrogen, —C₁-C₆ alkyl, —CH₂OH, or —OH;
R⁴ is hydrogen;
R⁵ is —(CH₂)ₙ—OH, —(CH₂)ₙNH₂, 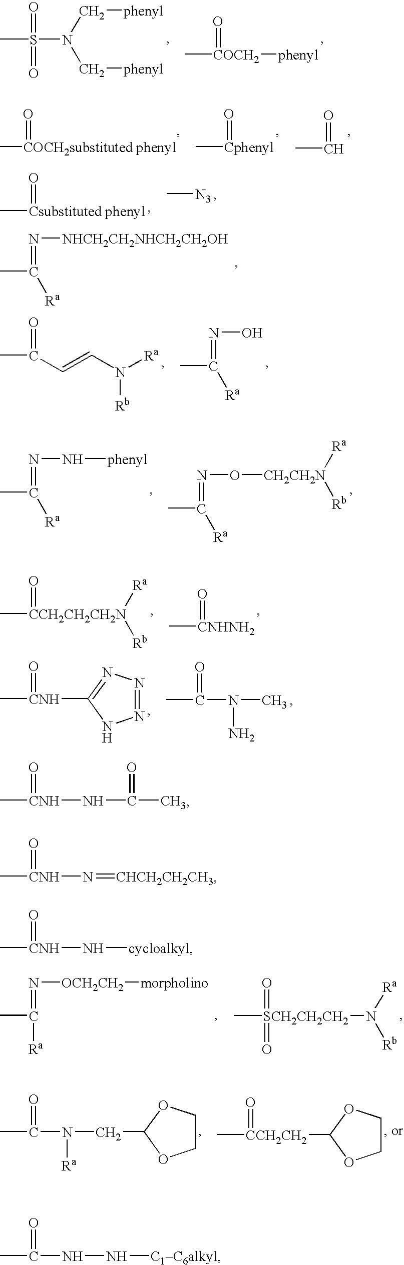
and the pharmaceutically acceptable salts thereof.

21. A coumpoun of claim 11 or claim 12 wherein R⁴ is in the para position.

22. A method of treating cancer, the method comprising administering to a patient having cancer, a therapeutically effective amount of a combination of:
1) 5-fluorouracil;
2) leucovorin; and
3) a compound of claim 11.

23. A method of treating cancer, the method comprising administering to a patient having cancer, a therapeutically effective amount of a combination of:
1) a thymidylate synthase inhibitor; and
2) a compound of claim 11.

24. The method of claim 22 wherein the cancer is colorectal cancer.

25. The method of claim 23 wherein the cancer is colorectal cancer.

26. The method of claim 23 wherein the thymidylate synthase inhibitor is 2-({5-[Methyl-(2-methyl-4-oxo-3,4-dihydro-quinazolin-6-ylmethyl)-amino]-thiophene-2-carbonyl}-amino)-pentanedioic acid.

27. The method of claim 23 wherein the thymidylate synthase inhibitor is
2-{4-[2-(2-Amino-4-oxo-4,7-dihydro-3H-pyrrolo[2,3-d]pyrimidin-5-yl)-ethyl]-benzoyl amino}-pentanedioic acid;
N⁶-Methyl-N⁶-[4-(morpholine-4-sulfonyl)-benzyl]-benzo[cd]indole-2,6-diamine; compound with 3,4,5,6-tetrahydroxy-tetrahydro-pyran-2-carboxylic acid; or
2-Amino-6-methyl-5-(pyridin-4-ylsulfanyl)-3H-quinazolin-4-one.

28. The method of claim 22 wherein the cancer is a P-glycoprotein positive cancer.

29. The method of claim 23 wherein the cancer is a P-glycoprotein positive cancer.

30. A pharmaceutical composition that comprises a compound of claim 11.

31. A method of treating cancer, the method comprising administering to a patient having cancer, a therapeutically effective amount of a combination of:
1) 5-fluorouracil;
2) leucovorin; and
3) a compound of Formula II 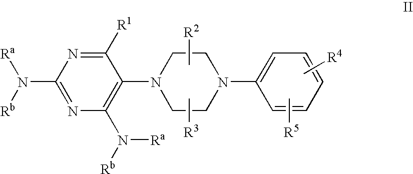
wherein
each R^{a} and R^{b} are independently hydrogen, C₁-C₆ alkyl, 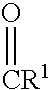
or C₁-C₆ cycloalkyl;
each n is hydrogen independently 0 to 5;
R¹ is hydrogen, C₁-C₆ alkyl, or —NR^{a}R^{b};
R² and R³ are independently hydrogen, C₁-C₆ alkyl, —CH₂OH, or —OH;
R⁴ is hydrogen, halogen, C₁-C₆ alkyl, —NO₂, —CN, CF₃, —OH, —OC₁-C₆
alkyl, —NR^{a}R^{b}, —CO₂R^{a}, or 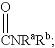
R⁵ is hydrogen, —CO₂R^{a}, 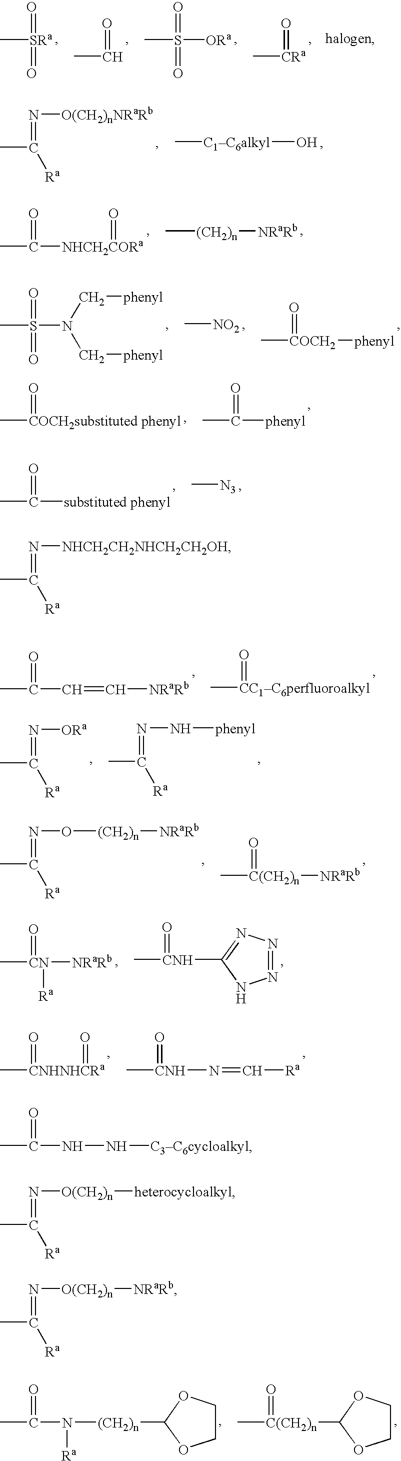 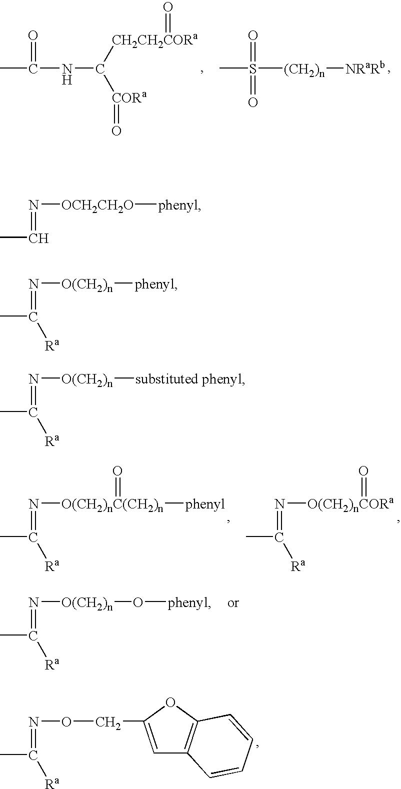
and the pharmaceutically acceptable salts thereof.

32. A method of treating cancer, the method comprising administering to a patient having cancer, a therapeutically effective amount of a combination of:
1) a thymidylate synthase inhibitor; and
2) a compound of Formula II 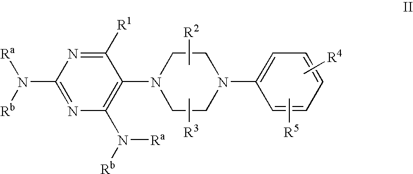
wherein
each R^{a} and R^{b} are independently hydrogen, C₁-C₆ alkyl, 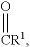
or C₁-C₆ cycloalkyl;
each n is independently 0 to 5;
R¹ is hydrogen, C₁-C₆ alkyl, or —NR^{a}R^{b};
R² and R³ are independently hydrogen, C₁-C₆ alkyl, —CH₂OH, or —OH;
R⁴ is hydrogen, halogen, C₁-C₆ alkyl, —NO₂, —CN, CF₃, —OH, —OC₁-C₆ alkyl, —NR^{a}R^{b}, —CO₂R^{a}, or 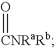
R⁵ is hydrogen, —CO₂R^{a}, 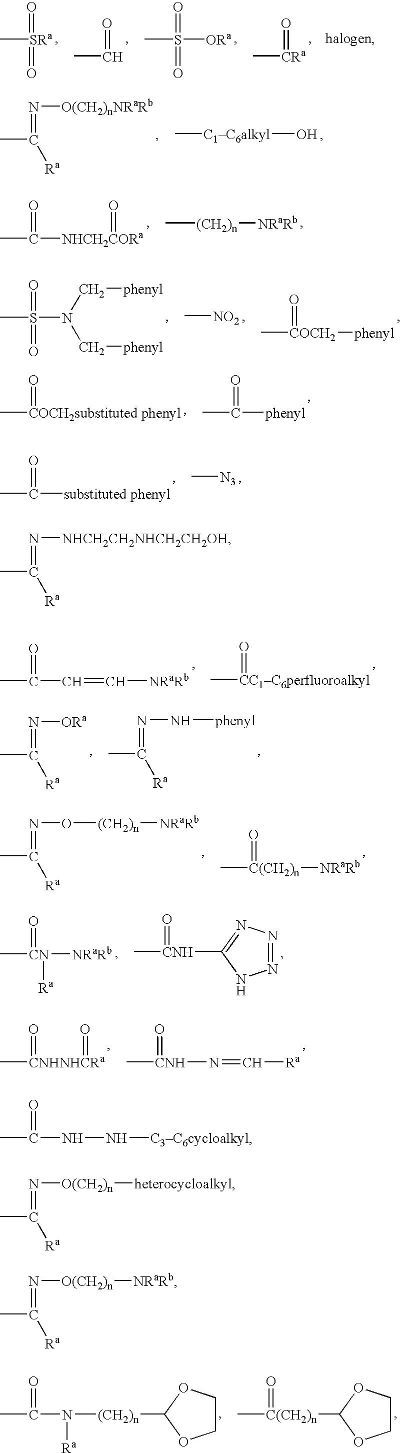 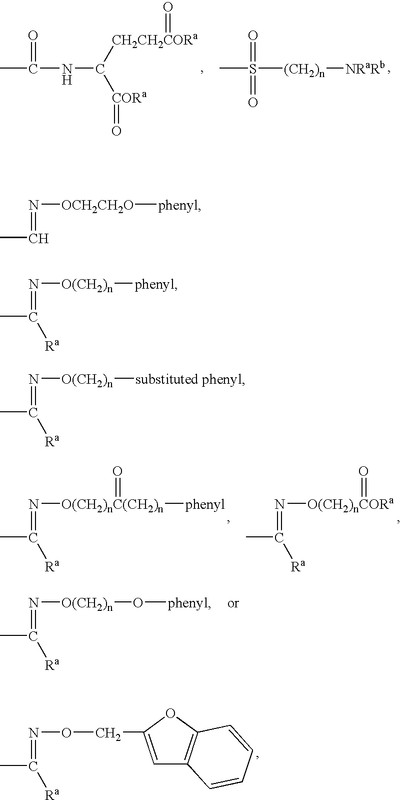
and the pharmaceutically acceptable salts thereof.

33. The method of claim 31 wherein the cancer is colorectal cancer.

34. The method of claim 32 wherein the cancer is colorectal cancer.

35. The method of claim 32 wherein the thymidylate synthase inhibitor is
2-({5-[Methyl-(2-methyl-4-oxo-3,4-dihydro-quinazolin-6-ylmethyl)-amino]-thiophene-2-carbonyl }-amino)-pentanedioic acid.

36. The method of claim 32 wherein the thymidylate synthase inhibitor is
2-{4-[2-(2-Amino-4-oxo-4,7-dihydro-3H-pyrrolo[2,3-d]pyrimidin-5-yl)-ethyl]-benzoylamino}-pentanedioic acid;
N⁶-Methyl-N⁶-[4-(morpholine-4-sulfonyl)-benzyl]-benzo[cd]indole-2,6-diamine; compound with 3,4,5,6-tetrahydroxy-tetrahydro-pyran-2-carboxylic acid; or
2-Amino-6-methyl-5-(pyridin-4-ylsulfanyl)-3H-quinazolin-4-one.

37. The method of claim 31 wherein the cancer is a P-glycoprotein positive cancer.

38. The method of claim 32 wherein the cancer is a P-glycoprotein positive cancer.
